(19) United States
(12) Patent Application Publication
(10) Pub. No.: US 2011/0233477 A1
(43) Pub. Date: Sep. 29, 2011

(54) Dye sensitised solar cell

(75) Inventors: Hiroshi Yamamoto, Nishinomiya-shi (JP); Shinji Nakamichi, Osaka (JP); Junichi Tanabe, Kasuya-gun (JP); Ryuichi Takahashi, Kobe-shi (JP); Hitoshi Yamato, Takarazuka-shi (JP)

(73) Assignee: BASF SE, Ludwigshafen (DE)

(21) Appl. No.: 13/073,251

(22) Filed: Mar. 28, 2011

Related U.S. Application Data
(60) Provisional application No. 61/318,394, filed on Mar. 29, 2010.

(51) Int. Cl. H01L 31/0224; C07D 401/14; C07D 213/80; C07D 401/10; C07D 455/04; C07D 413/10; C07D 401/06; H01B 1/22

(52) U.S. Cl. 252/501.1; 546/277.4; 546/322; 546/276.7; 546/94; 544/131; 540/591

(57) ABSTRACT

The present invention pertains to an electrode layer comprising a porous film made of oxide semiconductor fine particles sensitized with certain methine dyes. Moreover the present invention pertains to a photoelectric conversion device comprising said electrode layer, a dye sensitized solar cell comprising said photoelectric conversion device and to novel dyes of formula (I)

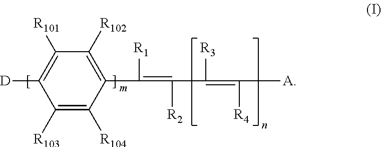

This application claims the benefit of U.S. Provisional Application No. 61/318,394, filed Mar. 29, 2010.

The present invention pertains to an electrode layer comprising a porous film made of oxide semiconductor fine particles sensitized with certain methine dyes. Moreover the present invention pertains to a photoelectric conversion device comprising said electrode layer, a dye sensitized solar cell comprising said photoelectric conversion device and to novel methine dyes.

CN-C-1253436, JP-A-2005/082678 and JP-A-2007/149570 disclose photoelectric conversion devices comprising some methine dyes.

US-A-2004/0260093 and WO-A-2006/097334 describe certain methine dyes, especially coumarin dyes, as optical markers for among others proteins.

It is the finding of the present invention that photoelectric conversion devices sensitized with certain methine dyes, i.e. methine dyes with a pyridinium acceptor group, whereby the acceptor group carries 2 anchoring groups one of which is attached to the pyridinium nitrogen, may have excellent overall properties, in particular that they may have a particularly good dye adsorption property onto TiO2 electrode, giving high long-term DSC cell stability and high long-term performance. Methine dyes with a 2-pyridinium acceptor group carrying 2 anchoring groups may be particularly advantageous.

The present invention pertains to an electrode layer comprising a porous film made of oxide semiconductor fine particles sensitized with a dye of formula (I),

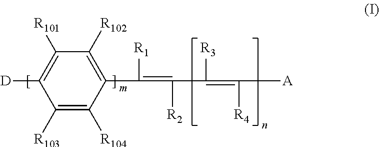

wherein
m is 0 or 1, preferably 0;
n is 0, 1, 2, 3, 4 or 5, preferably 0 or 1, most preferably 0;
R₁, R₂, R₃ and R₄ are independently H, —S(═O)₂OR₇, —S(═O)₂R₇, —S(═O)R₇, —S(═O)OR₇, fluorinated C₁-C₈alkyl, a group of formula (II)

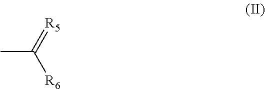

or unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof; or R₁ and R₂, or R₃ and R₄ form together an unsubstituted or substituted aliphatic 5-, 6- or 7-membered ring;
or if m is 0, R₁ and R₁₅, R₁ and R₁₆, R₂ and R₁₅ or R₂ and R₁₆ can form together an unsubstituted or substituted aliphatic 5-, 6- or 7-membered ring;
or if m is 0, R₂ and R₁₅ can form together an unsubstituted or substituted heteroaromatic 5-, 6- or 7-membered ring;
or a substituent R₃₀-R₃₃ of A ortho to the

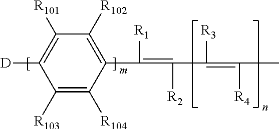

group and either if n is 0, R₂ or if n is 1, R₄ can form together an unsubstituted or substituted aliphatic 5-, 6- or 7-membered ring;

### or R₁ is D;

with the proviso that A is a group of formula (VI) or (VII) if at least one of R₁-R₄ is fluorinated C₁-C₈alkyl, —S(═O)₂OR₇, —S(═O)₂R₇, —S(═O)R₇, —S(═O)OR₇ or a group of formula (II)

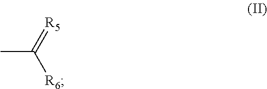

### R₅ is NR₈, N—OR₈, N—NR₈R₉, O or S;

R₆ is CO—SR₇, CO—NR₇—NR₁₀R₁₁, CO—NR₇—OR₁₀, CO—O—CO—R₇, CO—NR₇—CO—R₁₀, CO—NR₇—CO—OR₁₀, CO—NR₇—CO—NR₁₀R₁₁, NR₇R₁₀, OR₇, SR₇, NR₇—NR₁₀R₁₁, NR₇—OR₁₀, O—CO—R₇, O—CO—OR₇, O—CO—NR₇R₁₀, NR₇—CO—R₁₀, NR₇—CO—OR₁₀, NR₇—CO—NR₁₀R₁₁, CO—R₇, CO—OR₇, CO—NR₇R₁₀, NR₁₂—C(═NR₁₃)R₇ or unsubstituted or substituted C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₅-C₂₀aralkenyl, C₅-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
R₈ and R₉ are independently H or unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₅-C₂₀aralkenyl, C₅-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
R₇, R₁₀ and R₁₁ are independently H or unsubstituted or substituted C₁-C₂₀alkyl, C₂-C₂₀alkenyl, C₂-C₂₀alkynyl, C₆-C₂₀aryl, C₃-C₂₀heteroaryl, C₇-C₂₀aralkyl, C₅-C₂₀aralkenyl, C₅-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
or R₇ or R₁₀ together with that group of R₁-R₄ which is attached to the same double bond as the group of R₁-R₄ comprising said R₇ or R₁₀ forms an unsubstituted or substituted aliphatic 5-, 6- or 7-membered ring; or if R₇ or R₁₀ is part of R₂ with n being 0 or is part of R₄ with n being 1, it can form together with a substituent R₃₀-R₃₃ of A ortho to the

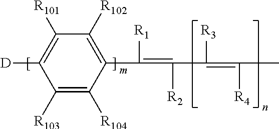

group an unsubstituted or substituted aliphatic 5-, 6- or 7-membered ring;
R₁₂ and R₁₃ form together an unsubstituted or substituted 5-, 6- or 7-membered ring;
R₁₄ is independently H or unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl;
D is independently a group of formula (III) or (IV)

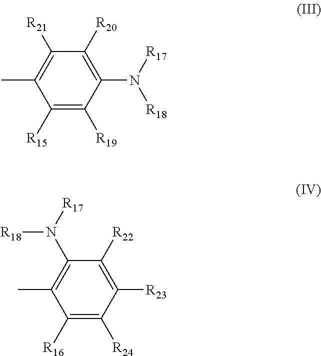

R₁₇ and R₁₈ are independently unsubstituted or substituted C₁-C₂₀alkyl, C₂-C₂₀alkenyl, C₂-C₂₀alkynyl, C₆-C₂₀aryl, C₄-C₃₆heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof; or R₁₇ and R₁₈, R₁₇ and R₂₂, R₁₇ and R₂₀ and/or R₁₈ and R₁₉ form together an unsubstituted or substituted 5-, 6- or 7-membered ring;
R₁₅, R₁₆, R₁₉, R₂₀, R₂₁, R₂₂, R₂₃ and R₂₄ are independently H, NR₂₅R₂₆, OR₂₅, SR₂₅, NR₂₅—NR₂₆R₂₇, NR₂₅—OR₂₆, O—CO—R₂₅, O—CO—OR₂₅, O—CO—NR₂₅R₂₆, NR₂₅—CO—R₂₆, NR₂₅—CO—OR₂₆, NR₂₅—CO—NR₂₆R₂₇, CO—R₂₅, CO—OR₂₅, CO—NR₂₅R₂₆, S—CO—R₂₅, CO—SR₂₅, CO—NR₂₅—NR₂₆R₂₇, CO—NR₂₅—OR₂₆, CO—O—CO—R₂₅, CO—O—CO—OR₂₅, CO—O—CO—NR₂₅R₂₆, CO—NR₂₅—CO—R₂₆, CO—NR₂₅—CO—OR₂₆, or unsubstituted or substituted C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
R₂₅, R₂₆ and R₂₇ are independently H or unsubstituted or substituted C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
A is a group of formula (V), (VI) or (VII)

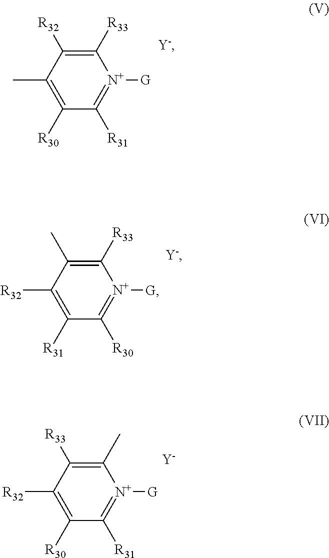

Y⁻ is an inorganic or organic anion, preferably Cl⁻, Br⁻, I⁻, SCN⁻, BF₄⁻, PF₆⁻, ClO₄⁻, SbF₆⁻, AsF₆⁻ or an organic anion selected from the group consisting of carboxylate, sulphonate, sulphate, phosphate, phosphonate, —CO(C═O)O⁻ and —NR₁₄—S(═O)₂O⁻;
G and G′ are independently —R₂₈—COOH, —R₂₈—COO⁻Z⁺, —R₂₈—CO(C═O)OH, —R₂₈—CO(C═O)O⁻Z⁺, —R₂₈—S(═O)₂OH, —R₂₈—S(═O)₂O⁻Z⁺, —R₂₈—O—S(═O)₂OH, —R₂₈—O—S(═O)₂O⁻Z⁺, —R₂₈—P(═O)(OH)₂, —R₂₈—P(═O)(O⁻Z⁺)₂, —R₂₈—P(═O)(OH)(O⁻Z⁺), —R₂₈—O—P(═O)(OH)₂, —R₂₈—O—P(═O)(O⁻Z⁺)₂, —R₂₈—O—P(═O)(OH)(O⁻Z⁺), —R₂₈—CO—NH—OH, —R₂₈—S(═O)₂NH—OH, —R₂₈—NR₁₄—S(═O)₂OH or —R₂₈—NR₁₄—S(═O)₂O⁻Z⁺;
Z⁺ is N(R₁₄)₄⁺, Li⁺, Na⁺ or K⁺ or is an ammonium cation which is part of a compound of formula (I) as part of group A;
R₂₈ is a direct bond or unsubstituted or substituted C₁-C₂₀alkylene, C₂-C₂₀alkenylene, C₂-C₂₀alkynylene, C₆-C₂₀arylene, C₄-C₉heteroarylene, C₇-C₁₁aralkylene, C₈-C₁₁aralkenylene, C₈-C₁₁aralkynylene, C₆-C₁₁heteroaralkylene, C₇-C₁₁heteroaralkenylene, C₇-C₁₁heteroaralkynylene, C₅-C₆cycloalkylene, C₁-C₂₀alkylene-C₆-C₂₀arylene, C₁-C₂₀alkylene-C₄-C₉heteroarylene, C₁-C₂₀alkylene-C₆-C₂₀arylene-C₁-C₂₀alkylene, C₁-C₂₀alkylene-C₄-C₉heteroarylene-C₁-C₂₀alkylene, —O—C₁-C₂₀alkylene, —O—C₂-C₂₀alkenylene, —O—C₂-C₂₀alkynylene, —O—C₆-C₂₀arylene, —O—C₄-C₉heteroarylene, —O—C₇-C₁₁aralkylene, —O—C₈-C₁₁aralkenylene, —O—C₈-C₁₁aralkynylene, —O—C₆-C₁₁ heteroaralkylene, —O—C₇-C₁₁ heteroaralkenylene, —O—C₇-C₁₁ heteroaralkynylene, —O—C₅-C₆cycloalkylene, —O—C₁-C₂₀alkylene-C₆-C₂₀arylene, —O—C₁-C₂₀alkylene-C₄-C₉heteroarylene, —O—C₁-C₂₀alkylene-C₆-C₂₀arylene-C₁-C₂₀alkylene or —O—C₁-C₂₀alkylene-C₄-C₉heteroarylene-C₁-C₂₀alkylene;
R₃₀, R₃₁, R₃₂ and R₃₃ are independently G′, H, halogen, unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof; or

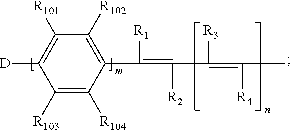

or two vicinal groups of R₃₀-R₃₃ form together an unsubstituted or substituted aliphatic 5-, 6- or 7-membered ring;
with the proviso that at least one of R₃₀-R₃₃ is G′;
R₁₀₁, R₁₀₂, R₁₀₃ and R₁₀₄ are independently H, halogen, unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;

### or R₁₀₂ or R₁₀₄ is D;

or R₁₀₁ and R₁₀₂ and/or R₁₀₃ and R₁₀₄ form together an unsubstituted or substituted aliphatic 5-, 6- or 7-membered ring;
or R₁₀₁ and R₁₅ or R₁₀₁ and R₁₆ form together an unsubstituted or substituted 5-, 6- or 7-membered ring.

For instance, R₂₈ is a straight chain group.

For example, n is 0 or 1, in particular 0;

for instance, R₁ is H or D;
for instance, R₂ is H, C₁-C₁₂alkyl (e.g. C₁-C₈alkyl) or C₆aryl;
for example, R₃ and R₄ are H;
for instance, if m is 0 and n is 0, R₁ is H and R₂ is H, C₁-C₁₂alkyl (e.g. C₁-C₈alkyl) or C₆aryl;
for example, if m is 0 and n is 1, R₁ is D and R₂, R₃ and R₄ are H;
for instance, if m is 1 and n is 0, R₁ and R₂ are H;
for instance, D is a group of formula (III);
for example, R₂₁ is H;
for instance, A is a group of formula (V) or (VII), in particular a group of formula (VII);
for example, Y⁻ is Br⁻, I⁻ or C₁-C₈alkyl-C₈aryl-S(═O)₂O⁻, or Y⁻ is an anionic group which is part of a compound of formula (I) and is selected from the group consisting of —O—S(═O)₂O⁻, COO⁻ and S(═O)₂O⁻, in particular Y⁻ is an anionic group which is part of a compound of formula (I) and is S(═O)₂O⁻;
for instance, G is —R₂₈—COOH, —R₂₈—COO⁻Z⁺, —R₂₈—CO(C═O)OH, —R₂₈—S(═O)₂OH, —R₂₈—S(═O)₂O⁻Z⁺, —R₂₈—CO—NH—OH or —R₂₈—S(═O)₂NH—OH, in particular —R₂₈—S(═O)₂O⁻Z⁺;
for example, G′ is —O—S(═O)₂O⁻Z⁺, —COOH or C₁-C₈alkylene-COOH, in particular —COOH or —CH₂—COOH, especially —COOH;
for instance, Z⁺ is the cationic group

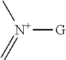

which is part or a compound of formula (I) as part of group A;
for example, R₂₈ is C₁-C₈alkylene, C₁-C₃alkylene-C₆arylene, —O—C₁-C₈alkylene or —O—C₁-C₃alkylene-C₆arylene, in particular C₁-C₄alkylene, C₁alkylene-C₆arylene, —O—C₁-C₄alkylene or —O—C₁alkylene-C₆arylene, especially C₁-C₄alkylene, such as C₃-C₄alkylene, e.g. C₃alkylene;
for instance, R₃₀ is G′;
for example, R₃₁, R₃₂ and R₃₃ are H;
for instance, if m is 1 and n is 0, R₁₀₁ and R₁₅ form together methylene, which is substituted by 2 C₁-C₈alkyl and R₁₀₂, R₁₀₃ and R₁₀₄ are H; or
for example, the compound of formula (I) is dimeric and R₂₁ is

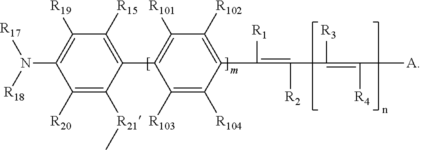

For example, m is 0 or 1;

n is 0 or 1;
R₁, R₂, R₃ and R₄ are independently H, —S(═O)₂OR₇, —S(═O)₂R₇, —S(═O)R₇, —S(═O)OR₇, fluorinated C₁-C₈alkyl, a group of formula (II), C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, fluorinated C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof;

### or R₁ is D;

or if m is 0, R₁ and R₁₅ or R₁ and R₁₆ can form together ethylene, trimethylene, tetramethylene, methylene-O, O-methylene, ethylene-O, O-ethylene, trimethylene-O, O-trimethylene, methylene-NR₁₄, NR₁₄-methylene, ethylene-NR₁₄, NR₁₄-ethylene, trimethylene-NR₁₄ or NR₁₄-trimethylene thus forming an aliphatic 5-, 6- or 7-membered ring;
or if m is 0, R₂ and R₁₅ can form together —O—, —S—, —C(O)— or —NR₁₄—;
or if m is 0 and D is a group of formula (IV), R₁ and R₁₈ can form together with the N-atom R₁₇ and R₁₈ are attached to

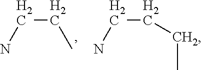

whereby in each of said groups one or more H-atom can be replaced by C₁-C₂₀alkyl, C₁-C₂₀alkylidene, phenyl or combinations thereof, in each of said groups two geminal H-atom can be replaced by C₁-C₂₀alkylidene, and/or in each of said groups two vicinal H-atoms can be replaced by benzo, trimethylene or tetramethylene;
with the proviso that A is a group of formula (VI) or (VII) if at least one of R₁-R₄ is fluorinated C₁-C₈alkyl, —S(═O)₂OR₇, —S(═O)₂R₇, —S(═O)R₇, —S(═O)OR₇ or a group of formula (II)

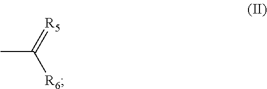

### R₅ is NR₈, N—OR₈, N—NR₈R₉, O or S;

R₆ is NR₇R₁₀, OR₇, SR₇, NR₇—NR₁₀R₁₁, NR₇—OR₁₀, O—CO—R₇, O—CO—OR₇, O—CO—NR₇R₁₀, NR₇—CO—R₁₀, NR₇—CO—OR₁₀, NR₇—CO—NR₁₀R₁₁, CO—R₇, CO—OR₇, CO—NR₇R₁₀, C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, fluorinated C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof;
R₈ and R₉ are independently H, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, fluorinated C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof;
R₇, R₁₀ and R₁₁ are independently H, C₁-C₂₀alkyl, C₂-C₂₀alkenyl, C₂-C₂₀alkynyl, C₆-C₂₀aryl, C₃-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, fluorinated C₁-C₂₀alkyl, fluorinated O—C₁-C₂₀alkyl, —CN, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₉-C₂₀cycloalkenyl, C₆-C₂₀cycloalkynyl or combinations thereof;
or R₇ or R₁₀ being part of R₂ forms together with R₁ a direct bond, methylene or ethylene thus forming an aliphatic 5-, 6- or 7-membered ring;
or R₇ or R₁₀ being part of R₄ forms together with R₃ a direct bond, methylene or ethylene thus forming an aliphatic 5-, 6- or 7-membered ring;
or if m is 0 and R₇ or R₁₀ is part of R₂, it can form together with R₁₅ or R₁₆ a direct bond or methylene thus forming an aliphatic 6- or 7-membered ring;
or if R₇ or R₁₀ is part of R₂ with n being 0 or is part of R₄ with n being 1, it can form together with a substituent R₃₀-R₃₃ of A ortho to the

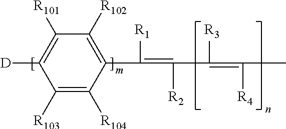

group a direct bond, methylene or ethylene thus forming an aliphatic 5-, 6- or 7-membered ring;
R₁₄, R₁₄′ are independently H, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl;
D is independently a group of formula (III) or (IV)
R₁₇ and R₁₈ are independently fluorenyl, C₁-C₂₀alkyl, C₂-C₂₀alkenyl, C₂-C₂₀alkynyl, C₆-C₂₀aryl, C₄-C₃₆heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the fluorenyl, alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by tetrahydrofuranyl, halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the fluorenyl, aryl and heteroaryl can be further substituted by maleic anhydridyl, maleimidyl, indenyl, C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof, whereby the maleic anhydridyl and maleimidyl are unsubstituted or substituted by C₁-C₂₀alkyl, C₆-C₂₀aryl, phenyl-NR₁₄R₁₄′ or combinations thereof;
or R₁₇ and R₁₈ form together with the N they are attached to piperidinyl, piperazinyl, morpholinyl, imidazolidinyl or pyrrollidinyl, each of which is unsubstituted or substituted by C₁-C₂₀alkyl, C₁-C₂₀alkylidene, benzo, trimethylene, tetramethylene or combinations thereof, which are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof;
or R₁₇ and R₂₂, R₁₇ and R₂₀ and/or R₁₈ and R₁₉ form together with the N-atom R₁₇ and R₁₈ are attached to

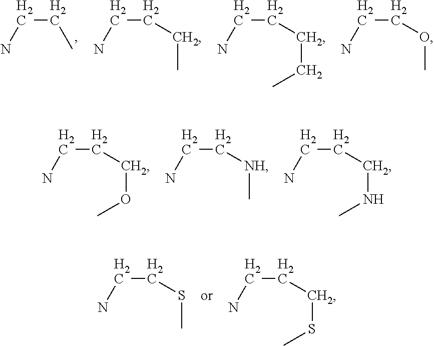

whereby in each of said groups one or more H-atom can be replaced by C₁-C₂₀alkyl, C₁-C₂₀alkylidene, phenyl, CO—OR₁₄, CONR₁₄R₁₄′ or combinations thereof, in each of said groups two geminal H-atom can be replaced by C₁-C₂₀alkylidene, and/or in each of said groups two vicinal H-atoms can be replaced by benzo, trimethylene or tetramethylene, whereby the benzo is unsubstituted or substituted by methyl (fluoren-9-ylidene);
R₁₅, R₁₆, R₁₉, R₂₀, R₂₁, R₂₂, R₂₃ and R₂₄ are independently H, NR₂₅R₂₆, OR₂₅, SR₂₅, NR₂₅—NR₂₆R₂₇, NR₂₅—OR₂₆, O—CO—R₂₅, O—CO—OR₂₅, O—CO—NR₂₅R₂₆, NR₂₅—CO—R₂₆, NR₂₅—CO—OR₂₆, NR₂₅—CO—NR₂₆R₂₇, CO—R₂₅, CO—OR₂₅, CO—NR₂₅R₂₆, CO—SR₂₅, CO—NR₂₅—NR₂₆R₂₇, CO—NR₂₅—OR₂₆, CO—O—CO—R₂₅, CO—O—CO—OR₂₅, CO—O—CO—NR₂₅R₂₆, CO—NR₂₅—CO—R₂₆, CO—NR₂₅—CO—OR₂₆, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof;
R₂₅, R₂₆ and R₂₇ are independently H, C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by pyridinium*Y⁻, maleic anhydridyl, maleimidyl, halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, C₂-C₂₀alkenyl, C₂-C₂₀alkynyl, C₇-C₂₀aralkyl, C₅-C₂₀aralkenyl, C₅-C₂₀aralkynyl, C₄-C₂₀cycloalkyl, C₅-C₂₀cycloalkenyl, C₆-C₂₀cycloalkynyl or combinations thereof, whereby the pyridinium, maleic anhydridyl, maleimidyl are unsubstituted or substituted by C₁-C₂₀alkyl, C₂-C₂₀alkenyl, C₂-C₂₀alkynyl, C₆-C₂₀aryl, C₆-C₂₀aryl-O—R₁₄, C₇-C₂₀aralkyl, C₅-C₂₀aralkenyl, C₅-C₂₀aralkynyl, C₄-C₂₀cycloalkyl, C₅-C₂₀cycloalkenyl, C₆-C₂₀cycloalkynyl, S—R₁₄, O—R₁₄, CO—OR₁₄, O—CO—R₁₄, NR₁₄R₁₄′, CONR₁₄R₁₄′, NR₁₄—CO—R₁₄′ or combinations thereof;
A is a group of formula (V), (VI) or (VII),
Y⁻ is Cl⁻, Br⁻, I⁻, SCN⁻, BF₄⁻, PF₆⁻, ClO₄⁻, SbF₆⁻, AsF₆⁻ or an organic anion selected from the group consisting of C₁-C₂₀alkyl-COO⁻, C₆-C₂₀aryl-COO⁻, C₁-C₂₀alkyl-S(═O)₂O⁻, C₆-C₂₀aryl-S(═O)₂O⁻, C₁-C₂₀alkyl-O—S(═O)₂O⁻, C₆-C₂₀aryl-O—S(═O)₂O⁻, C₁-C₂₀alkyl-P(═O)₂O⁻, C₆-C₂₀aryl-P(═O)₂O⁻, C₁-C₂₀alkyl-O—P(═O)₂O⁻ and C₆-C₂₀aryl-O—P(═O)₂O⁻, whereby the aryl is unsubstituted or substituted by 1 to 4 C₁-C₂₀alkyl, or Y⁻ is an anionic group which is part of a compound of formula (I) and is selected from the group consisting of COO⁻, S(═O)₂O⁻, O—S(═O)₂O⁻, P(═O)(O⁻)(O⁻Z⁺), P(═O)(OH)(O⁻), O—P(═O)(O⁻)(O⁻Z⁺), O—P(═O)(OH)(O⁻), —CO(C═O)O⁻ and —NR₁₄—S(═O)₂O⁻;
G and G′ are independently —R₂₈—COOH, —R₂₈—COO⁻Z⁺, —R₂₈—CO(C═O)OH, —R₂₈—CO(C═O)O⁻Z⁺, —R₂₈—S(═O)₂OH, —R₂₈—S(═O)₂O⁻Z⁺, —R₂₈—O—S(═O)₂OH, —R₂₈—O—S(═O)₂O⁻Z⁺, —R₂₈—P(═O)(OH)₂, —R₂₈—P(═O)(O⁻Z⁺)₂, —R₂₈—P(═O)(OH)(O⁻Z⁺), —R₂₈—O—P(═O)(OH)₂, —R₂₈—O—P(═O)(O⁻Z⁺)₂, —R₂₈—O—P(═O)(OH)(O⁻Z⁺), —R₂₈—CO—NH—OH, —R₂₈—S(═O)₂NH—OH, —R₂₈—NR₁₄—S(═O)₂OH or —R₂₈—NR₁₄—S(═O)₂O⁻Z⁺;
Z⁺ is N(R₁₄)₄⁺, Li⁺, Na⁺ or K⁺ or is the cationic group

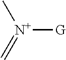

which is part of a compound of formula (I) as part of group A;
R₂₈ is a direct bond or C₁-C₂₀alkylene, C₂-C₂₀alkenylene, C₂-C₂₀alkynylene, C₆-C₂₀arylene, C₄-C₉heteroarylene, C₇-C₁₁aralkylene, C₅-C₁₁aralkenylene, C₅-C₁₁aralkynylene, C₆-C₁₁heteroaralkylene, C₇-C₁₁heteroaralkenylene, C₇-C₁₁heteroaralkynylene, C₅-C₆cycloalkylene, C₁-C₂₀alkylene-C₆-C₂₀arylene, C₁-C₂₀alkylene-C₄-C₉heteroarylene, C₁-C₂₀alkylene-C₆-C₂₀arylene-C₁-C₂₀alkylene, C₁-C₂₀alkylene-C₄-C₉heteroarylene-C₁-C₂₀alkylene, —O—C₁-C₂₀alkylene, —O—C₂-C₂₀alkenylene, —O—C₂-C₂₀alkynylene, —O—C₆-C₂₀arylene, —O—C₄-C₉heteroarylene, —O—C₇-C₁₁aralkylene, —O—C₈-C₁₁aralkenylene, —O—C₈-C₁₁aralkynylene, —O—C₆-C₁₁heteroaralkylene, —O—C₇-C₁₁heteroaralkenylene, —O—C₇-C₁₁heteroaralkynylene, —O—C₅-C₆cycloalkylene, —O—C₁-C₂₀alkylene-C₆-C₂₀arylene, —O—C₁-C₂₀alkylene-C₄-C₉heteroarylene, —O—C₁-C₂₀alkylene-C₆-C₂₀arylene-C₁-C₂₀alkylene or —O—C₁-C₂₀alkylene-C₄-C₉heteroarylene-C₁-C₂₀alkylene, whereby each of said groups is unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the arylene and heteroarylene can be further substituted by 1-4 C₁-C₂₀alkyl;
R₃₀, R₃₁, R₃₂ or R₃₃ are independently G′, H, halogen, pyridinium*Y⁻, quinolinium*Y⁻, isoquinolinium*Y⁻, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl, whereby the whereby the pyridinium, quinolinium, isoquinolinium, alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by 1-4 C₁-C₂₀alkyl, and whereby the pyridinium, quinolinium and isoquinolinium can be further substituted by G′, C₁-C₂₀alkyl or combinations thereof; or

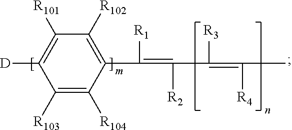

or two vicinal groups of R₃₀-R₃₃ form together trimethylene, tetramethylene or pentamethylene, each of which is unsubstituted or substituted by G′, benzo, R₁₄ or combinations thereof.
with the proviso that at least one of R₃₀-R₃₃ is G′;
R₁₀₁, R₁₀₂, R₁₀₃ and R₁₀₄ are independently H, halogen, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof;

### or R₁₀₂ or R₁₀₄ is D;

or R₁₀₁ and R₁₅ or R₁₀₁ and R₁₆ form together methylene, ethylene, ethenylene or trimethylene, whereby in each of said groups one or more H-atom can be replaced by C₁-C₂₀alkyl, C₁-C₂₀alkylidene, phenyl or combinations thereof, in each of said groups two geminal H-atom can be replaced by C₁-C₂₀alkylidene, and/or in each of said groups two vicinal H-atoms can be replaced by benzo, trimethylene or tetramethylene;
or the compound of formula (I) is dimeric and R₁₈ is

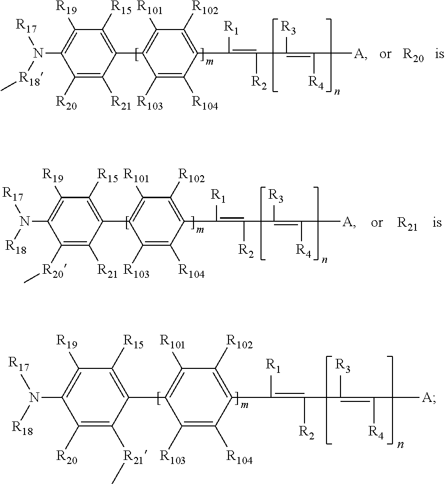

### R₁₈′ is R₃₇;

R₂₀′ and R₂₁′ are R₃₇, O—R₃₇—O, S—R₃₇—S, NR₁₄—R₃₇—NR₁₄′, CO—R₃₇—CO, CO—O—R₃₇—O—CO, CO—NR₁₄—R₃₇—NR₁₄′-CO, CO—S—R₃₇—S—CO, O—CO—R₃₇—CO—O, NR₁₄—CO—R₃₇—CO—NR₁₄′ or S—CO—R₃₇—CO—S;
R₃₇ is C₁-C₂₀alkylene, C₂-C₂₀alkenylene, C₂-C₂₀alkynylene, C₆-C₂₀arylene, C₄-C₂₀heteroarylene, C₇-C₂₀aralkylene, C₈-C₂₀aralkenylene, C₈-C₂₀aralkynylene, C₄-C₂₀cycloalkylene, C₁-C₈alkylene-C₆-C₂₀arylene-C₁-C₈alkylene (e.g.

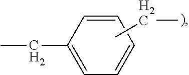

C₅-C₂₀cycloalkenylene or C₆-C₂₀cycloalkynylene, whereby the alkylene and cycloalkylene is uninterrupted or interrupted by O, S, C(═O), NR₁₄ or combinations thereof, and whereby the alkylene, alkenylene, alkynylene, arylene, heteroarylene, aralkylene, aralkenylene, aralkynylene, cycloalkylene, alkylene-arylene-alkylene, cycloalkenylene and cycloalkynylene are unsubstituted or substituted by fluorine, and whereby the arylene, heteroarylene and aryl can be further substituted by C₁-C₂₀alkyl, fluorinated C₁-C₂₀alkyl or combinations thereof, and the remainder of the substituents are as defined above.

For instance, m is 0 or 1;

n is 0 or 1;
R₁, R₂, R₃ and R₄ are independently H, C₁-C₂₀alkyl or C₆-C₂₀aryl;

### or R₁ is D;

R₁₄, R₁₄′ are independently H, C₁-C₁₄alkyl, C₆aryl or C₇-C₁₀aralkyl;
D is a group of formula (III);
R₁₇ and R₁₈ are independently C₁-C₁₄alkyl, C₂-C₈alkenyl, C₆-C₂₀aryl, C₄-C₃₆heteroaryl, C₇-C₂₀aralkyl, C₈-C₂₀aralkenyl, C₈-C₁₀aralkynyl or C₅-C₁₂cycloalkyl, whereby the alkyl, alkenyl, aryl, heteroaryl, aralkyl, aralkenyl, aralkynyl and cycloalkyl, are unsubstituted or substituted by tetrahydrofuranyl, halogen, S—R₁₄, O—R₁₄, CO—OR₁₄, O—CO—R₁₄, NR₁₄R₁₄′, CONR₁₄R₁₄′, NR₁₄—CO—R₁₄′ or combinations thereof, and the aryl and heteroaryl can be further substituted by C₁-C₈alkyl, C₂-C₈alkenyl or C₈-C₂₀aralkenyl;
or R₁₇ and R₁₈ form together with the N they are attached to morpholinyl;
or R₁₇ and R₂₀ and/or R₁₅ and R₁₉ form together with the N-atom R₁₇ and R₁₅ are attached to

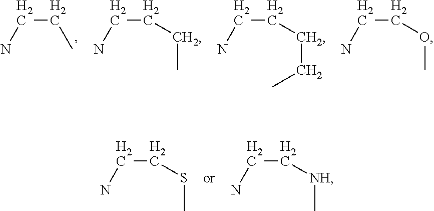

whereby in each of said groups one or more H-atom can be replaced by C₁-C₂₀alkyl, C₆-C₂₀aryl, CO—OR₁₄, CONR₁₄R₁₄′ or combinations thereof, in each of said groups two geminal H-atom can be replaced by C₁-C₂₀alkylidene, and/or in each of said groups two vicinal H-atoms can be replaced by benzo, trimethylene or tetramethylene, whereby the benzo is unsubstituted or substituted by methyl (fluoren-9-ylidene);

### R₁₅ is H, NR₂₅R₂₆, OR₂₅, SR₂₅, O—CO—R₂₅ or NR₂₅—CO—R₂₆;

### R₁₉, R₂₀ and R₂₁ are H;

R₂₅ and R₂₆ are independently H, C₁-C₁₄alkyl, C₆aryl or C₇-C₁₀aralkyl, whereby the alkyl, aryl and aralkyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′ or combinations thereof;
A is a group of formula (V) or (VII),
Y⁻ is Cl⁻, Br⁻, I⁻, SCN⁻, BF₄⁻, PF₆⁻, ClO₄⁻, SbF₆⁻, AsF₆⁻ or an organic anion selected from the group consisting of, C₁-C₂₀alkyl-S(═O)₂O⁻, C₆-C₂₀aryl-S(═O)₂O⁻, C₁-C₂₀alkyl-O—S(═O)₂O⁻, C₆-C₂₀aryl-O—S(═O)₂O⁻, C₁-C₂₀alkyl-P(═O)₂O⁻, C₆-C₂₀aryl-P(═O)₂O⁻, C₁-C₂₀alkyl-O—P(═O)₂O⁻ and C₆-C₂₀aryl-O—P(═O)₂O⁻, whereby the aryl is unsubstituted or substituted by 1 to 4 C₁-C₂₀alkyl, or Y⁻ is an anionic group which is part of a compound of formula (I) and is selected from the group consisting of COO⁻, CO(C═O)O⁻, S(═O)₂O⁻ or OS(═O)₂O⁻;
G and G′ are independently —R₂₈—COOH, —R₂₈—COO⁻Z⁺, —R₂₈—CO(C═O)OH, —R₂₈—CO(C═O)O⁻Z⁺, —R₂₈—S(═O)₂OH, —R₂₈—S(═O)₂O⁻Z⁺, —R₂₈—OS(═O)₂OH, —R₂₈—OS(═O)₂O⁻Z⁺, —R₂₈—CO—NH—OH or —R₂₈—S(═O)₂NH—OH;
Z⁺ is N(R₁₄)₄⁺, Li⁺, Na⁺ or K⁺ or is the cationic group

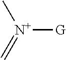

which is part of a compound of formula (I) as part of group A;
R₂₈ is a direct bond or C₁-C₂₀alkylene, C₁-C₂₀alkylene-C₆-C₂₀arylene, —O—C₁-C₂₀alkylene or —O—C₁-C₂₀alkylene-C₆-C₂₀arylene;

### R₃₀ is G′;

### R₃₁, R₃₂ and R₃₃ are H;

R₁₀₁ and R₁₅ form together methylene, which is substituted by 1 or 2 C₁-C₂₀alkyl;

### R₁₀₂, R₁₀₃ and R₁₀₄ are H;

or the compound of formula (I) is dimeric and R₂₁ is

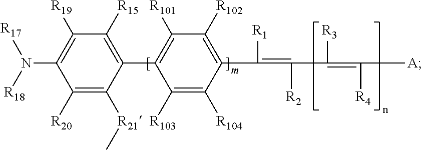

R₂₁′ is O—C₁-C₂₀alkylene-O; and the remainder of the substituents are as defined above.

For instance, m is 0 or 1;

n is 0 or 1;
R₁, R₂, R₃ and R₄ are independently H, C₁-C₁₂alkyl or C₆aryl;

### or R₁ is D;

D is a group of formula (III);
R₁₇ and R₁₈ are independently C₁-C₁₄alkyl, C₆aryl, C₄-C₃₀heteroaryl, whereby the aryl is substituted by C₈-C₂₀aralkenyl;
or R₁₇ and R₁₈ form together with the N they are attached to morpholinyl;
or R₁₇ and R₂₀ and/or R₁₈ and R₁₉ form together with the N-atom R₁₇ and R₁₈ are attached to

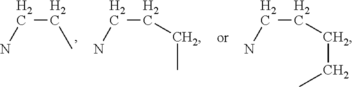

whereby in each of said groups one or more H-atom can be replaced by C₁-C₈alkyl, CON(C₁-C₁₂alkyl)₂, or in each of said groups two vicinal H-atoms can be replaced by benzo or trimethylene;
R₁₅ is H or O—C₁-C₁₄alkyl;

### R₁₉, R₂₀ and R₂₁ are H;

A is a group of formula (VII),
Y⁻ is Br⁻, I⁻ or C₁-C₈alkyl-C₆aryl-S(═O)₂O⁻, or Y⁻ is an anionic group which is part of a compound of formula (I) and is selected from the group consisting of COO⁻, OS(═O)₂O⁻ and S(═O)₂O⁻;
G is —R₂₈—COOH, —R₂₈—COO⁻Z⁺, —R₂₈—S(═O)₂OH, —R₂₈—S(═O)₂O⁻Z⁺, —R₂₈—CO—NH—OH or —R₂₈—S(═O)₂NH—OH;

### G′ is —COOH, —CO—COOH, —CH₂—COOH or —OS(═O)₂O⁻Z⁺;

Z⁺ is the cationic group

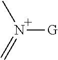

which is part of a compound of formula (I) as part of group A;
R₂₈ is C₁-C₈alkylene, C₁-C₃alkylene-C₆arylene, —O—C₁-C₈alkylene or —O—C₁-C₃alkylene-C₆arylene;

### R₃₀ is G′;

### R₃₁, R₃₂ and R₃₃ are H;

R₁₀₁ and R₁₅ form together methylene, which is substituted by 2 C₁-C₁₂alkyl;

### R₁₀₂, R₁₀₃ and R₁₀₄ are H;

or the compound of formula (I) is dimeric and R₂₁ is

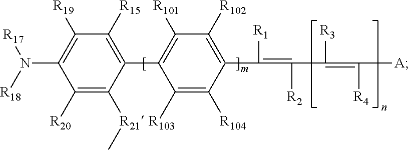

R₂₁′ is O—C₁-C₈alkylene-O; and the remainder of the substituents are as defined above.

Preferably, m is 0;

n is 0;

### R₁ and R₂ are H;

D is a group of formula (III);
R₁₇ and R₁₈ are independently C₁-C₁₄alkyl, C₆aryl, C₄-C₃₀heteroaryl, whereby the aryl is substituted by C₈-C₂₀aralkenyl;
or R₁₇ and R₂₀ form together with the N-atom R₁₇ and R₁₈ are attached to

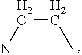

whereby in said group two vicinal H-atoms can be replaced by tetramethylene or trimethylene, whereby the tetramethylene can be substituted by cyclohexanonyl and in the trimethylene two vicinal H-atoms can be replaced by benzo;
R₁₅ is H or O—C₁-C₁₄alkyl;

### R₁₉, R₂₀ and R₂₁ are H;

A is a group of formula (VII),
Y⁻ is an anionic group which is part of a compound of formula (I) and is selected from the group consisting of COO⁻ and S(═O)₂O⁻;

### G is —R₂₈—COOH or —R₂₈—S(═O)₂O⁻Z⁺;

### G′ is —COOH or —COO⁻Z⁺;

Z⁺ is the cationic group

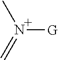

which is part of a compound of formula (I) as part of group A;
R₂₈ is C₁-C₈alkylene;

### R₃₀ is G′;

### R₃₁, R₃₂ and R₃₃ are H.

For example, m is 0;

n is 0;

### R₁ and R₂ are H;

D is a group of formula (III);
R₁₇ and R₁₈ are independently C₁-C₁₄alkyl or C₄-C₃₀heteroaryl;
or R₁₈ and R₁₉ form together with the N-atom R₁₇ and R₁₈ are attached to

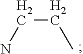

R₁₅ is H or O—C₁-C₁₄alkyl;

### R₁₉, R₂₀ and R₂₁ are H;

A is a group of formula (VII),
Y⁻ is an anionic group which is part of a compound of formula (I) and is S(═O)₂O⁻;

### G is —R₂₈—S(═O)₂O⁻Z⁺;

### G′ is —COOH;

Z⁺ is the cationic group

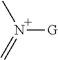

which is part of a compound of formula (I) as part of group A;
R₂₈ is C₁-C₈alkylene, preferably C₁-C₅alkylene, especially C₃alkylene, in particular n-C₃alkylene;

### R₃₀ is G′;

### R₃₁, R₃₂ and R₃₃ are H.

In compounds of formula (I)

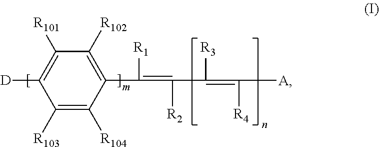

D is a donor moiety, A is an acceptor moiety and

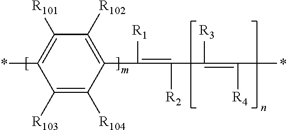

is a spacer moiety. So compounds of formula (I) contain a donor, a spacer and an acceptor.

* indicates a free valence.

Some examples of donors D are:

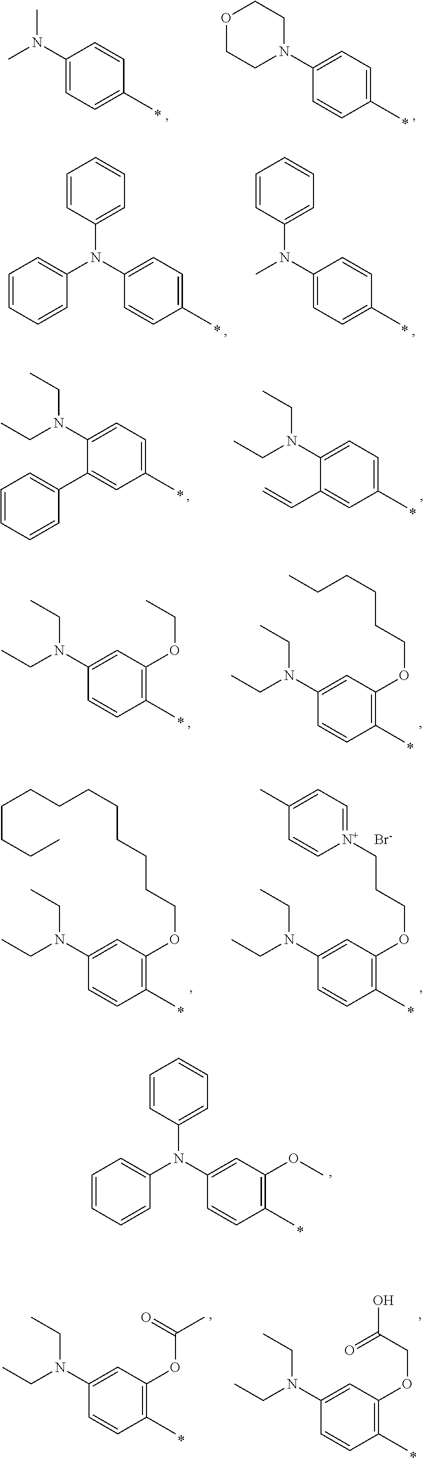

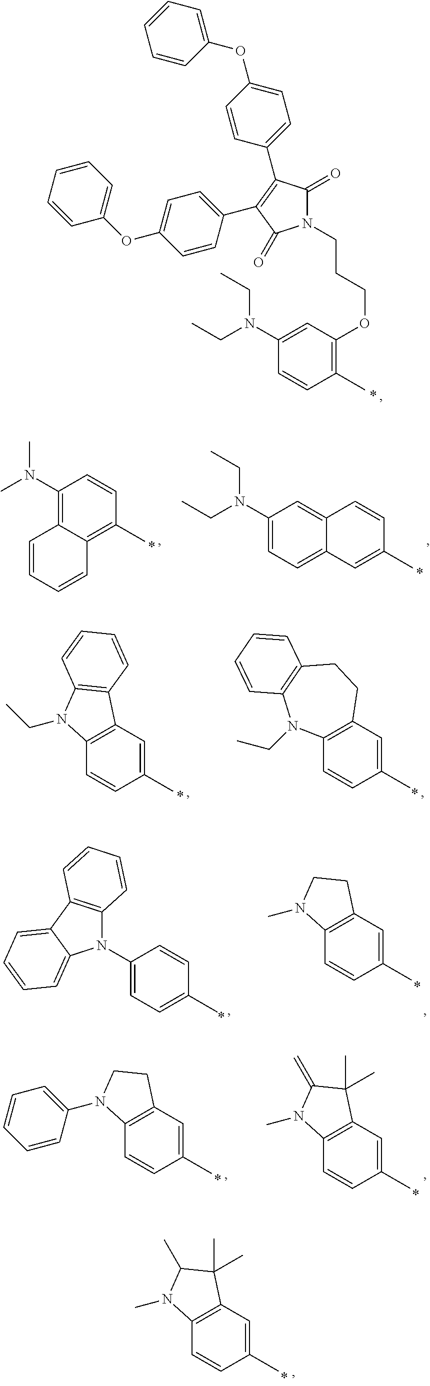

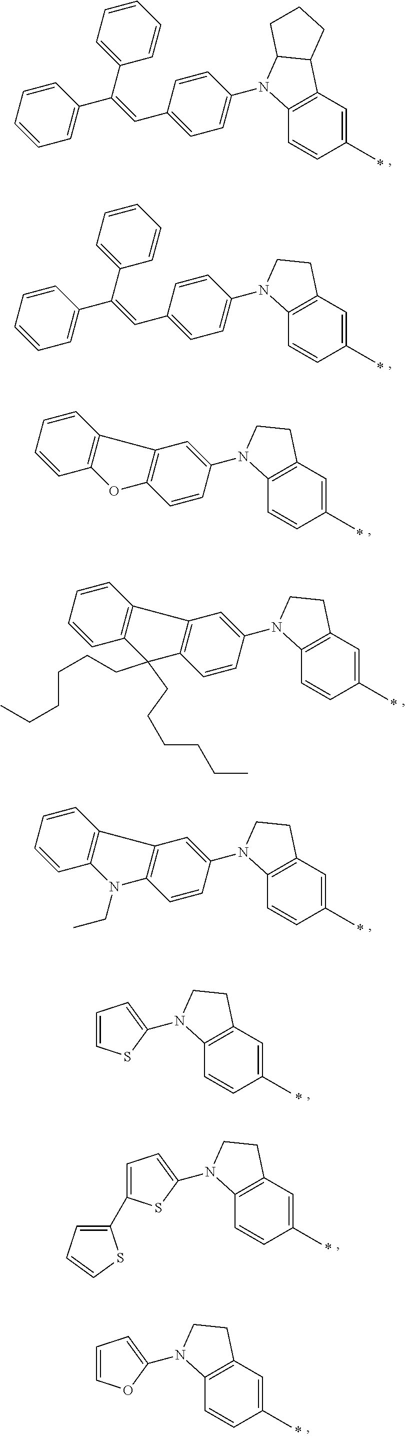

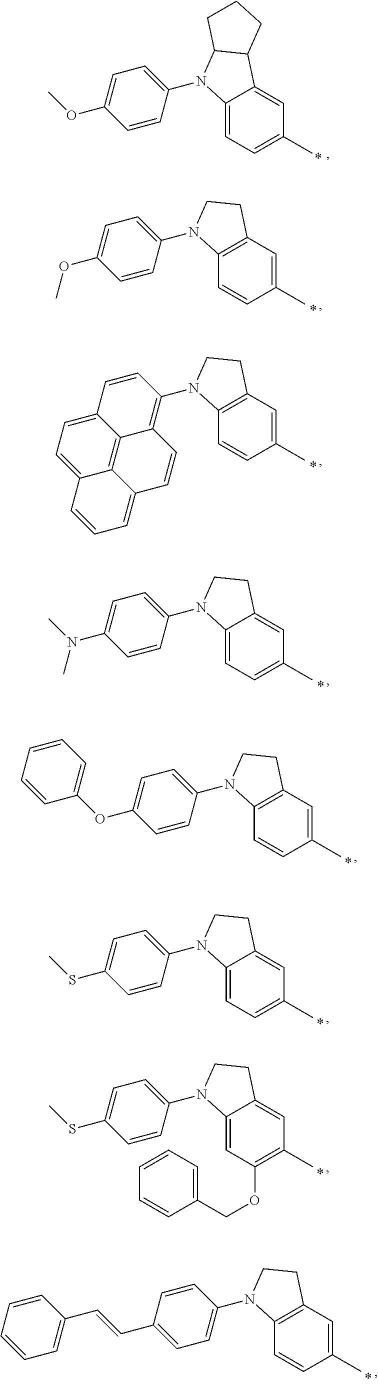

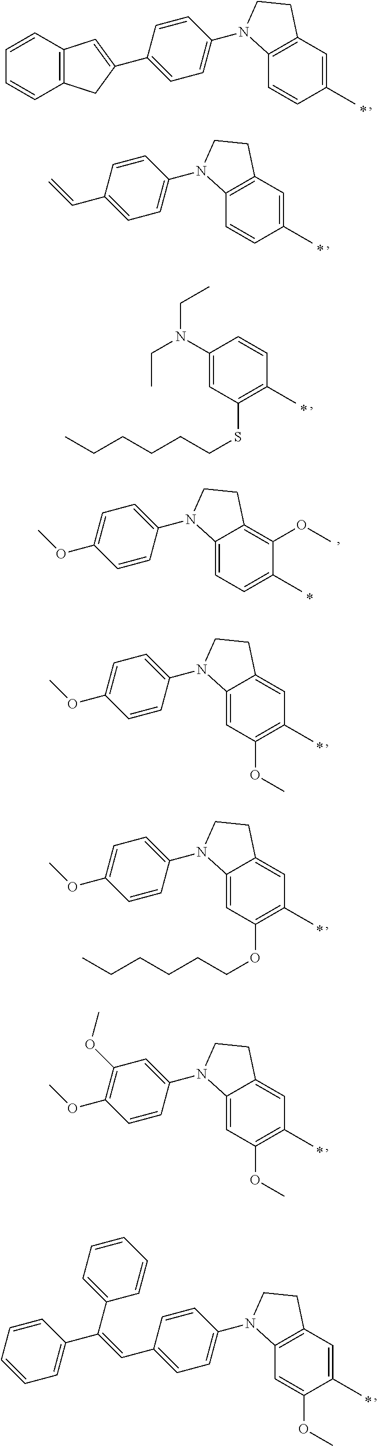

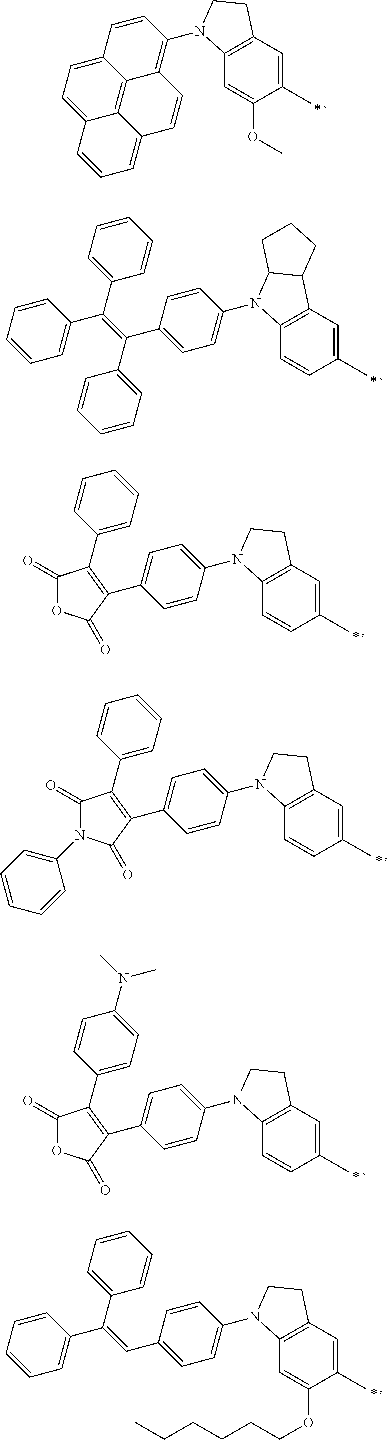

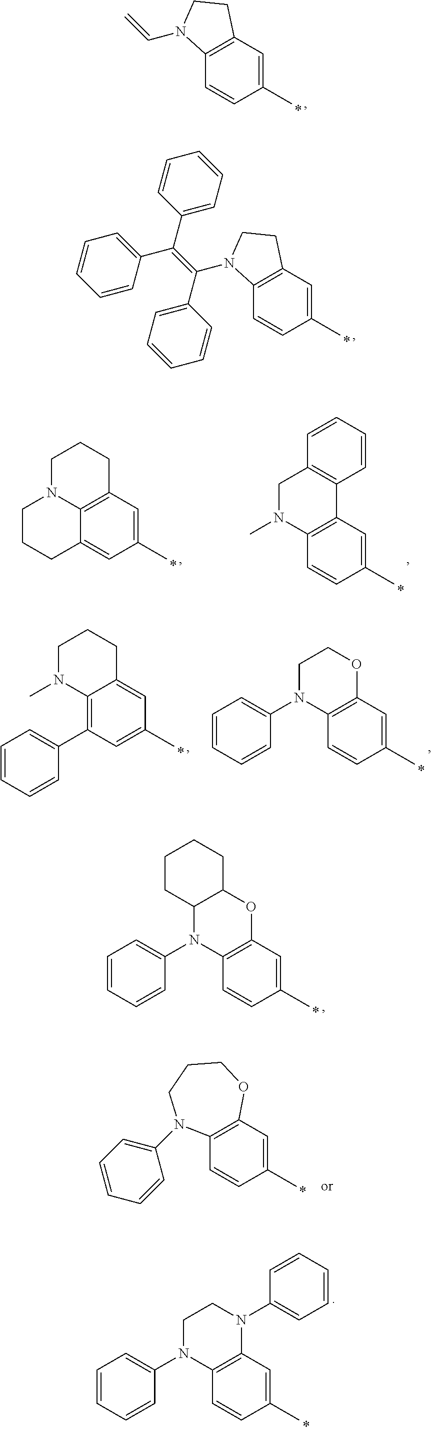

Examples of dimeric donors D are

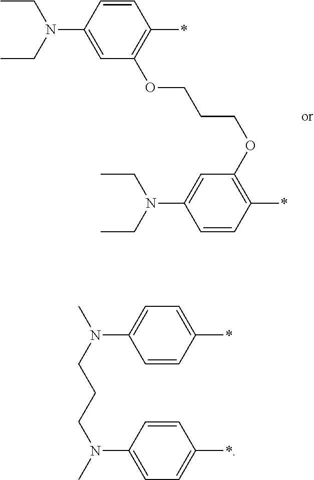

Particularly useful compounds of formula (I) are as listed below:

| Com- | | | |
|---|---|---|---|
| pound | | | |
| No. | Donor | Spacer | Acceptor |
| C1 | 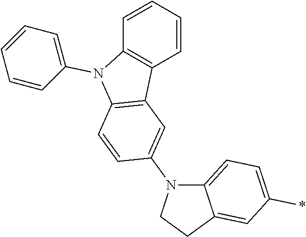 | 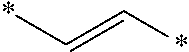 | 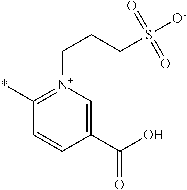 |
| C2 | 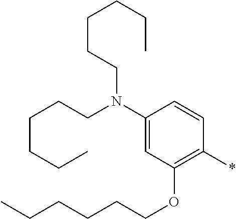 | 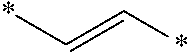 | 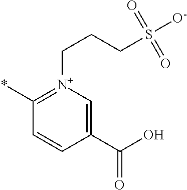 |
| C3 | 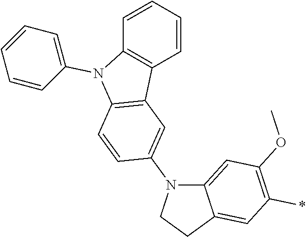 | 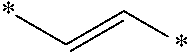 | 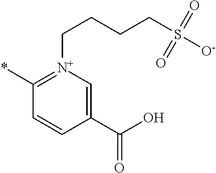 |
| C4 | 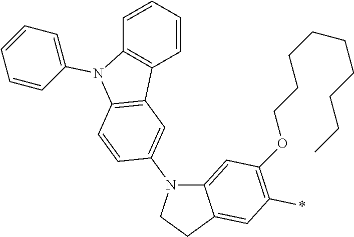 | 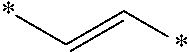 | 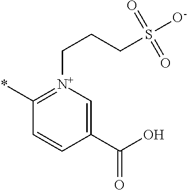 |
| C5 | 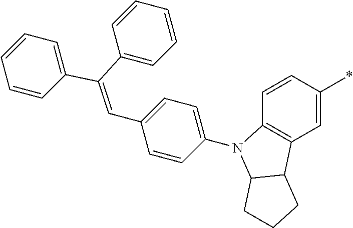 | 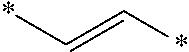 | 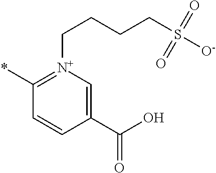 |
| C6 | 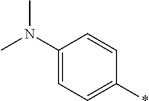 | 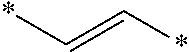 | 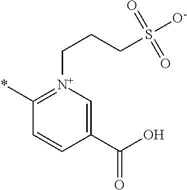 |
| C7 | 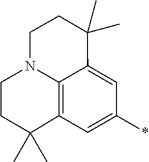 | 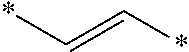 | 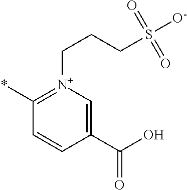 |
| C8 | 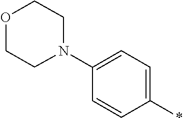 | 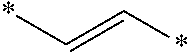 | 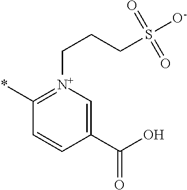 |
| C9 | 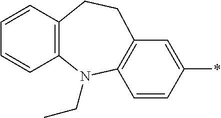 | 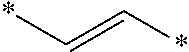 | 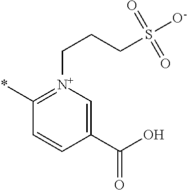 |
| C10 | 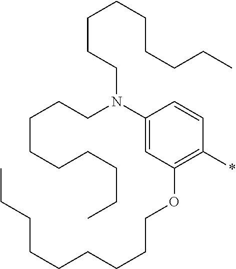 | 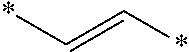 | 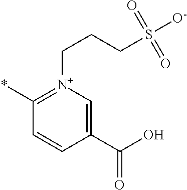 |
| C11 | 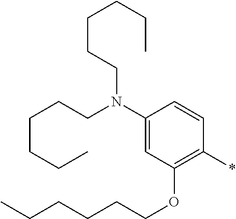 | 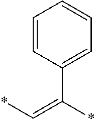 | 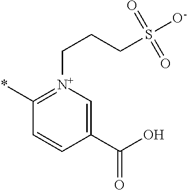 |
| C12 | 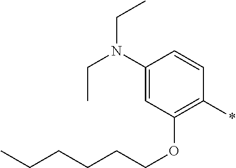 | 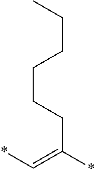 | 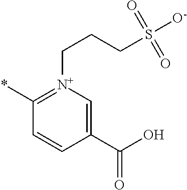 |
| C13 | 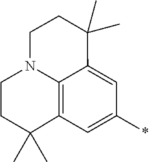 | 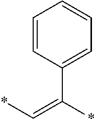 | 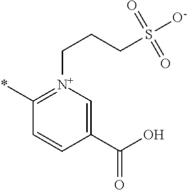 |
| C14 | 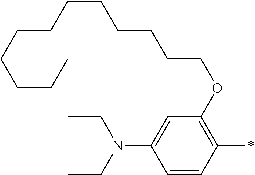 | 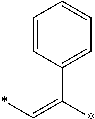 | 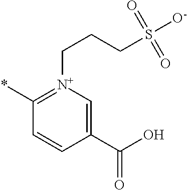 |
| C15 | 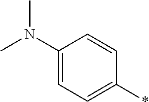 | 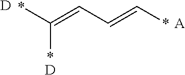 | 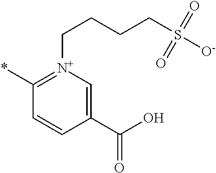 |
| C16 | 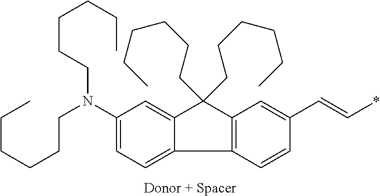 | | 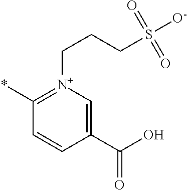 |
| C17 | 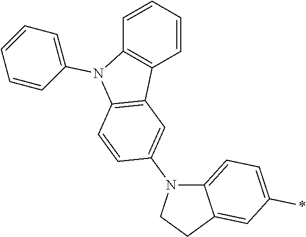 | 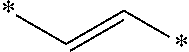 | 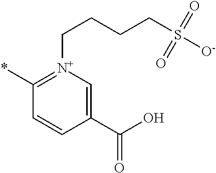 |
| C18 | 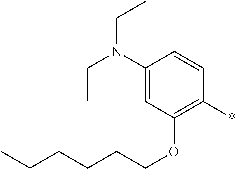 | 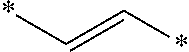 | 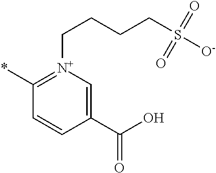 |
| C19 | 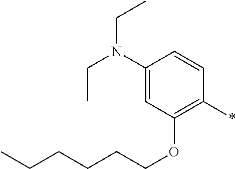 | 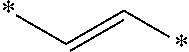 | 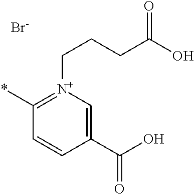 |
| C20 | 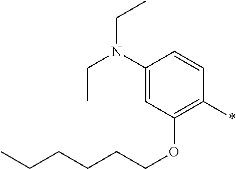 | 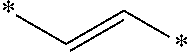 | 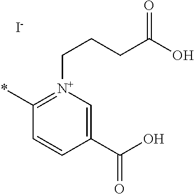 |
| C21 | 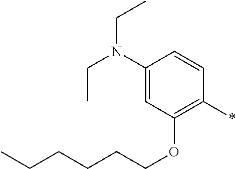 | 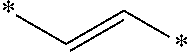 | 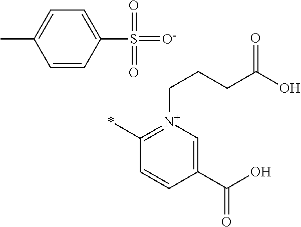 |
| C22 | 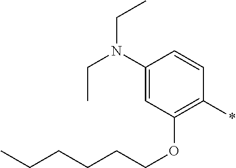 | 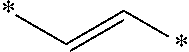 | 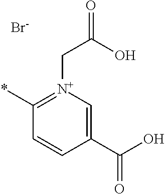 |
| C23 | 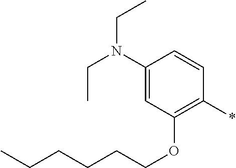 | 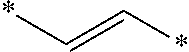 | 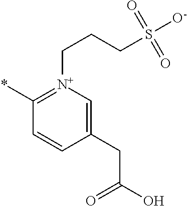 |
| C24 | 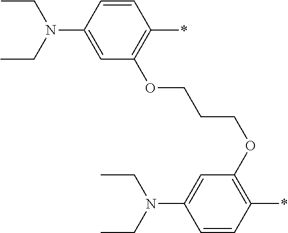 | 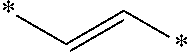 | 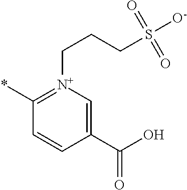 |
| C25 | 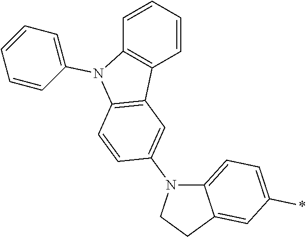 | 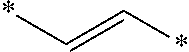 | 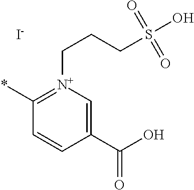 |
| C26 | 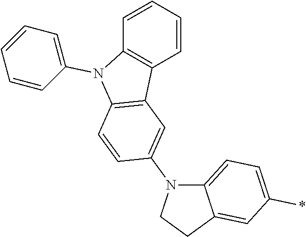 | 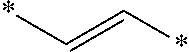 | 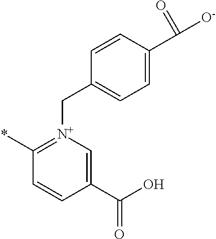 |
| C27 | 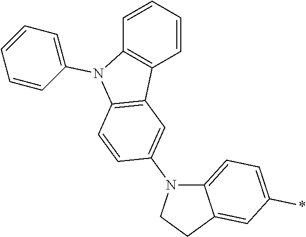 | 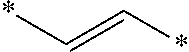 | 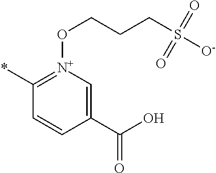 |
| C28 | 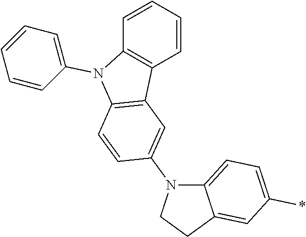 | 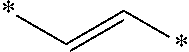 | 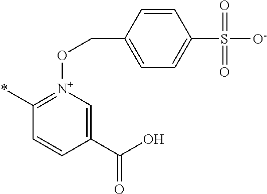 |
| C29 | 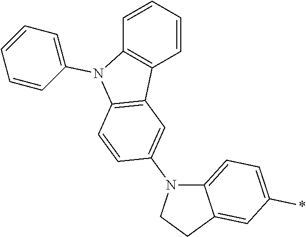 | 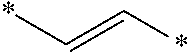 | 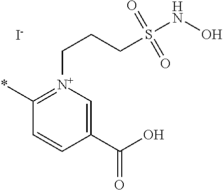 |
| C30 | 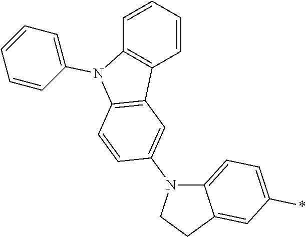 | 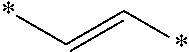 | 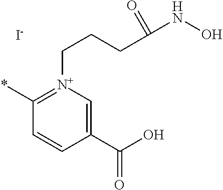 |
| C31 | 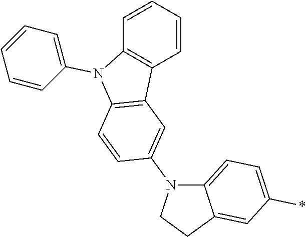 | 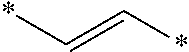 | 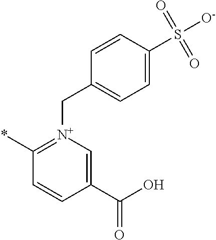 |
| C32 | 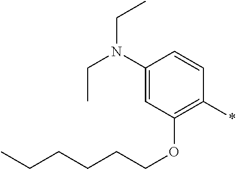 | 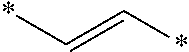 | 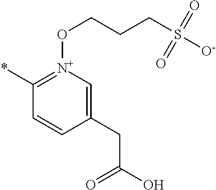 |
| C33 | 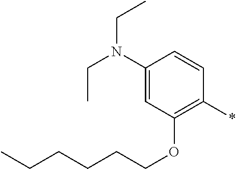 | 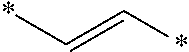 | 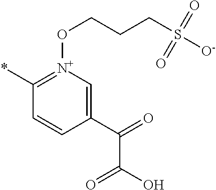 |
| C34 | 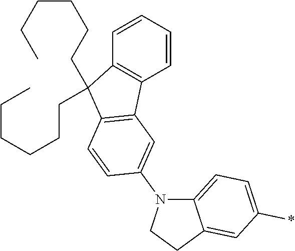 | 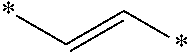 | 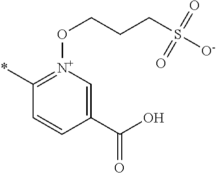 |
| C35 | 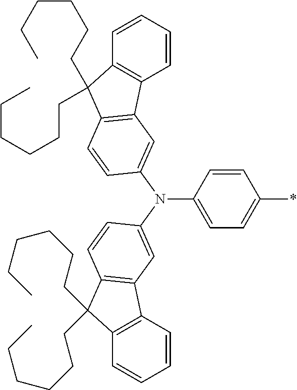 | 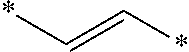 | 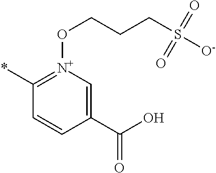 |
| C36 | 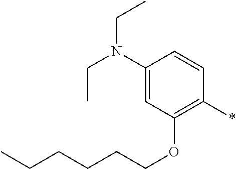 | 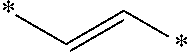 | 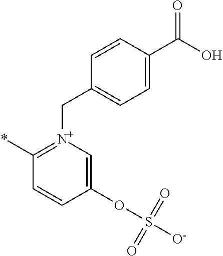 |
| C37 | 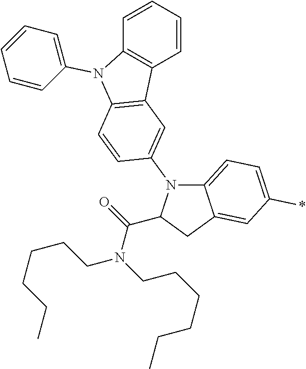 | 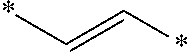 | 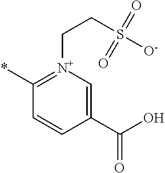 |
| C38 | 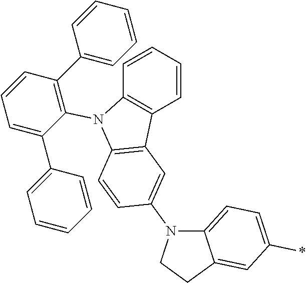 | 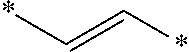 | 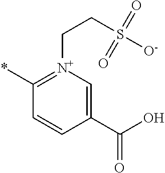 |
| C39 | 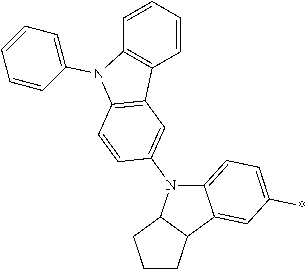 | 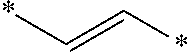 | 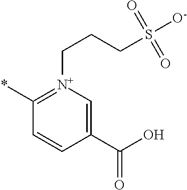 |
| C40 | 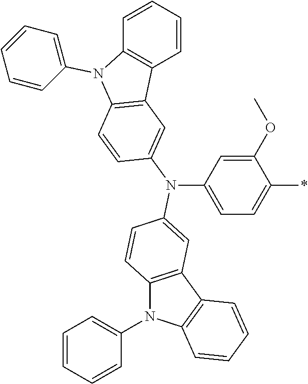 | 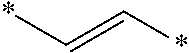 | 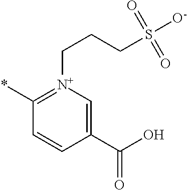 |
| C41 | 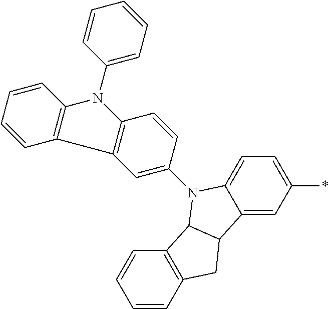 | 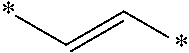 | 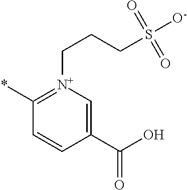 |
| C42 | 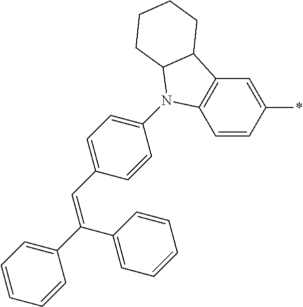 | 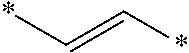 | 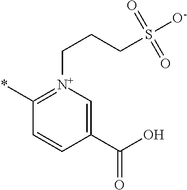 |
| C43 | 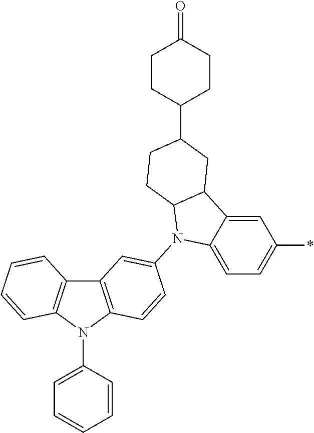 | 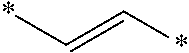 | 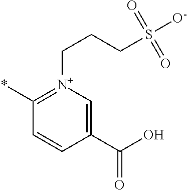 |
| C44 | 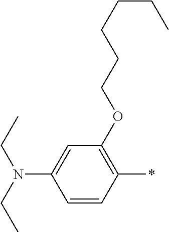 | 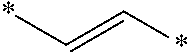 | 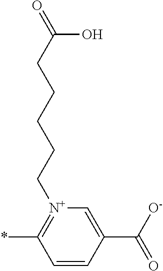 |
| C45 | 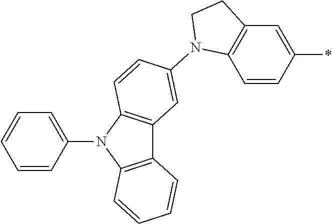 | 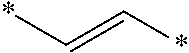 | 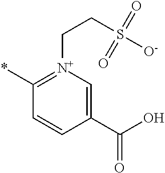 |

The oxide semiconductor fine particles are, for instance, made of TiO₂, SnO₂, WO₃, ZnO, Nb₂O₅, Fe₂O₃, ZrO₂, MgO, WO₃, ZnO, CdS, ZnS, PbS, Bi₂S₃, CdSe, CdTe or combinations thereof, preferably made of TiO₂.

For instance, the electrode layer comprises a dye of formula (I) or a mixture of dyes of formula (I) as the only dye(s).

Preferred is a porous film made of oxide semiconductor fine particles which is sensitized with a dye of formula (I) and one or more further dyes.

Examples of further dyes are metal complex dyes (preferably the metal is Ru, Pt, Ir, Rh, Re, Os, Fe, W, Cr, Mo, Ni, Co, Mn, Zn or Cu, more preferably Ru, Os or Fe, most preferably Ru) and/or organic dyes selected from the group consisting of indoline, courmarin, cyanine, merocyanine, hemicyanine, methine, azo, quinone, quinonimine, diketo-pyrrolo-pyrrole, quinacridone, squaraine, triphenylmethane, perylene, indigo, xanthene, eosin, rhodamine and combinations thereof. As further dyes organic dyes, in particular methine dyes are preferred. For instance, the further dye is different from dyes of formula (I).

For instance, the molar ratio of a further dye to a dye of formula (I) is 1:19 to 19:1, preferably 1:9 to 9:1, more preferably 1:5 to 5:1, most preferably 1:3 to 3:1.

For example, the dye is adsorbed together with an additive, preferably a co-adsorbent.

Examples of such additives are co-adsorbents selected from the group consisting of a steroid (preferably deoxycholic acid, dehydrodeoxcholic acid, chenodeoxycholic acid, cholic acid methyl ester, cholic acid sodium salt or combinations thereof), a sulfonate, a carboxylic acid, a phosphinic acid, a crown ether, a cyclodextrine, a calixarene, a polyethyleneoxide and combinations thereof, especially a steroid such as chenodeoxycholic acid.

Examples of co-adsorbent are:

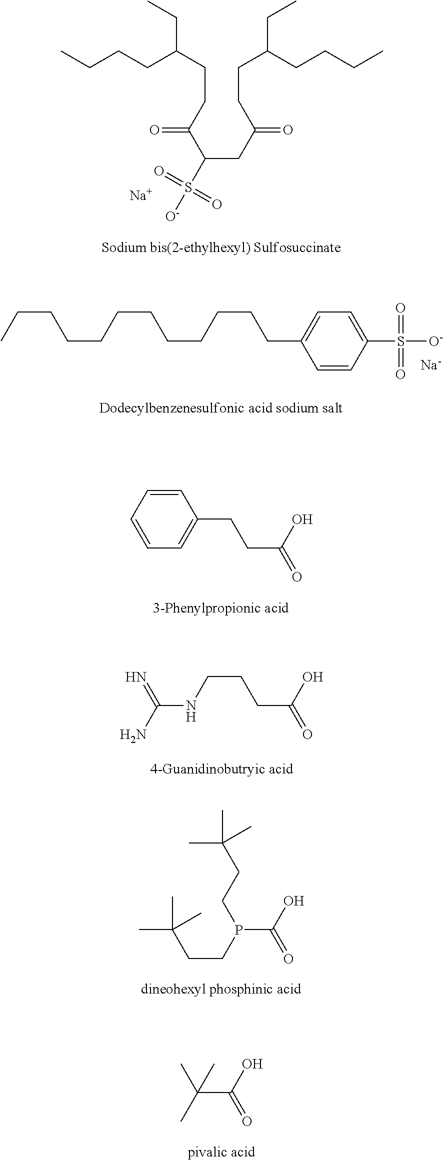

For example, the molar ratio of such an additive to a dye of formula (I) is 1000:1 to 1:100, preferably 100:1 to 1:10, most preferably 10:1 to 1:2.

For example, such an additive is not a dye.

The present invention also pertains to a photoelectric conversion device comprising an electrode layer as defined herein.

Such photoelectric conversion devices usually comprise

(a) a transparent conductive electrode substrate layer,
(b) an electrode layer comprising a porous film made of oxide semiconductor fine particles sensitized with
(c) a dye of formula (I),
(d) a counter electrode layer, and
(e) an electrolyte layer (e.g. filled between the working electrode layer b and the counter electrode layer d).

The component (c) can also be a combination of a dye of formula (I) and one or more further dyes.

Preferably, the transparent conductive electrode substrate layer (a) contains (e.g. consists of)

(a-1) a transparent insulating layer and
(a-2) a transparent conductive layer.

The transparent conductive layer (a-2) is usually between the transparent insulating layer (a-1) and the electrode layer (b).

Examples of the transparent insulating layer (a-1) include glass substrates of soda glass, fused quartz glass, crystalline quartz glass, synthetic quartz glass; heat resistant resin sheets such as a flexible film; metal sheets, transparent plastic sheets made of polyethylene terephthalate (PET), polyethylene naphthalate (PEN), polycarbonate (PC), polyether sulfone (PES); a polished plate of a ceramic, such as titanium oxide or alumina.

Examples of transparent conductive layer (a-2) are conductive metal oxides such as ITO (indium-tin compounded oxide), IZO (indium-zinc compounded oxide), FTO (fluorine-doped tin oxide), zinc oxide doped with boron, gallium or aluminum, and niobium-doped titanium oxide. The thickness of the transparent conductive layer (a-2) is usually 0.1 to 5 p.m. The surface resistance is usually below 40 ohms/sq, preferably below 20 ohms/sq.

To improve the conductivity of the transparent conductive layer (a-2), it is possible to form a metal wiring layer on it, made of for instance silver, platinum, aluminum, nickel or titanium. The area ratio of the metal wiring layer is generally within the range that does not significantly reduce the light transmittance of the transparent conductive electrode substrate layer (a). When such a metal wiring layer is used, the metal wiring layer may be provided as a grid-like, stripe-like, or comb-like pattern.

The electrode layer (b) is usually between the transparent conductive electrode substrate layer (a) and the electrolyte layer (e).

The porous film of oxide semiconductor fine particles of the electrode layer (b) can be prepared by a hydrothermal process, a sol/gel process or high temperature hydrolysis in gas phase. The fine particles usually have an average particle diameter of from 1 nm to 1000 nm. Particles with different size can be blended and can be used as either single or multi-layered porous film. The porous film of the oxide semiconductor layer (b) has usually a thickness of from 0.5 to 50 p.m.

If desired, it is possible to form a blocking layer on the electrode layer (b) (usually between the surface of the electrode layer (b) and the dye (c)) and/or between the electrode layer (b) and the trans-parent conductive electrode substrate layer (a) to improve the performance of the electrode layer (b). An example of forming a blocking layer is immersing the electrode layer (b) into a solution of metal alkoxides such as titanium ethoxide, titanium isopropoxide and titanium butoxide, chlorides such as titanium chloride, tin chloride and zinc chloride, nitrides and sulfides and then drying or sintering the substrate. For instance, the blocking layer is made of a metal oxide (e.g. TiO₂, SiO₂, Al₂O₃, ZrO₂, MgO, SnO₂, ZnO, Eu₂O₃, Nb₂O₅ or combinations thereof) or a polymer (e.g. poly(phenylene oxide-co-2-allylphenylene oxide) or poly(methylsiloxane)). Details of the preparation of such layers are described in, for example, Electrochimica Acta, 1995, 40, 643; J. Phys. Chem. B, 2003, 107, 14394; J. Am. Chem. Soc., 2003, 125, 475; Chem. Lett, 2006, 35, 252; J. Phys. Chem. B, 2006, 110, 19191; J. Phys. Chem. B, 2001, 105, 1422. The blocking layer may be applied to prevent undesired reaction. The blocking is usually dense and compact, and is usually thinner than the electrode layer (b).

Preferably, the counter electrode layer (d) contains (e.g. consists of)
(d-1) a conductive layer and
(d-2) an insulating layer.

The conductive layer (d-1) is usually between the insulating layer (d-2) and the electrolyte layer (e).

For instance, the conductive layer (d-1) contains a conductive carbon (e.g. graphite, single walled carbon nanotubes, multiwalled carbon nanotubes, carbon nanofibers, carbon fibers, grapheme or carbon black), a conductive metal (e.g. gold or platinum), a metal oxide (e.g. ITO (indium-tin compounded oxide), IZO (indium-zinc compounded oxide), FTO (fluorine-doped tinoxide), zinc oxide doped with boron, gallium or aluminum, and niobium-doped titanium oxide) or mixtures thereof.

Furthermore, the conductive layer (d-1) may be one obtained by forming a layer of platinum, carbon or the like (generally with a thickness of from 0.5 to 2,000 nm), on a thin film of a conductive oxide semiconductor, such as ITO, FTO, or the like (generally with a thickness of from 0.1 to 5 μm). The layer of platinum, carbon or the like is usually between the electrolyte layer (e) and the insulating layer (d-2).

Examples of the insulating layer (d-2) includes glass substrates of soda glass, fused quartz glass, crystalline quartz glass, synthetic quartz glass; heat resistant resin sheets such as a flexible film; metal sheets, transparent plastic sheets made of polyethylene terephthalate (PET), polyethylene naphthalate (PEN), polycarbonate (PC), polyether sulfone (PES); a polished plate of a ceramic, such as titanium oxide or alumina.

The dye (c) is usually disposed on the electrode layer (b) on that surface of the electrode layer (b) facing the electrolyte layer (e).

For adsorption of the dye (c) to the electrode layer (b), the electrode layer (b) may be immersed into a solution or a dispersion liquid of the dye. A concentration of the dye solution or dye dispersion liquid is not limited to, but preferably from 1 μM to 1M, and is preferably 10 μM to 0.1M. The time period for the dye adsorption is preferably from 10 seconds to 1000 hours, more preferably from 1 minute to 200 hours, most preferably from 1 to 10 hours. The temperature for dye adsorption is preferably from room temperature to the boiling temperature of the solvent or the dispersion liquid. The adsorption may be carried out dipping, immersing or immersing with stirring. As the stirring method, a stirrer, supersonic dispersion, a ball mill, a paint conditioner, a sand mill or the like is employed, while the stirring method shall not be limited thereto.

The solvent for dissolving or dispersing the dye (c) includes water, alcohol solvents such as methanol, ethanol, isopropyl alcohol, t-butyl alcohol, ethylene glycol and propylene glycol, ether solvents such as dioxane, diethyl ether, dimethoxyethane, tetrahydrofuran, dioxolane, t-butyl methyl ether, ethylene glycol dialkyl ether, propylene glycol monomethyl ether acetate and propylene glycol methyl ether, ketone solvents such as acetone, amide solvents such as N,N-dimethylformamide, N,N-dimethylacetamide and N-methylpyrrolidone, nitrile solvents such as acetonitrile, methoxy acetonitrile, methoxy propionitrile, propionitrile and benzonitrile, carbonate solvents such as ethylene carbonate, propylene carbonate and diethyl carbonate, heterocyclic compounds such as 3-methyl-2-oxazolidinone, dimethyl sulfoxide, sulfolane and γ-butyrolactone, halogenated hydrocarbon solvents such as dichloromethane, chloroform, dichloroethane, trichloroethane, trichloroethylene, chlorobenzene, o-dichlorobenzene, 1-chloronaphthalene, bromoform, bromobenzene, methyl iodide, iodobenzene and fluorobenzene and hydrocarbon solvents such as benzene, toluene, o-xylene, m-xylene, p-xylene, ethylbenzene, cumene, n-pentane, n-hexane, n-octane, cyclohexane, methylcyclohexane, 1,5-hexadiene and cyclohexadiene. These may be used solely or in the form of a mixture containing two or more solvents. As a solvent, supercritical solvent such as supercritical carbon dioxide may be used.

As dye (c) a dye of formula (I) may be adsorbed on the electrode layer (b) solely or in combination with one or more further dyes. The dyes adsorbed together are not limited to dyes of formula (I). Two or more dyes may be adsorbed on the electrode layer (b) one by one or all together by dissolving the dyes in a solvent. It is preferable to use the dyes with different absorption peaks in different wavelengths to absorb wide range of light wavelengths and generate higher current. The ratio of two or more dyes adsorbed on the electrode layer (b) is not limited but preferably each dye has molar ratio of more than 10%.

For adsorption of the dye (c), an additive may be used in combination. The additive may be any one of an agent that has a function presumably for controlling dye adsorption. The additive includes a condensation agent such as thiol or a hydroxyl compound and a co-adsorbent. These may be used solely or a mixture of them. The molar ratio of the additive to the dye is preferably 0.01 to 1,000, more preferably 0.1 to 100.

For instance, the dye-adsorbed electrode layer may be treated with amines such as 4-tert-butyl pyridine. As a treatment method, immersing the dye-sensitized electrode layer into amine solution which may be diluted with a solvent such as acetonitrile or ethanol can be employed.

In the above manner, the electrode layer of the present invention can be obtained.

When the electrolyte layer (e) is in the form of solution or quasi-solid, the electrolyte layer (e) usually contains,

(e-1) electrolyte compound,
(e-2) solvent and/or ionic liquid, and
preferably (e-3) other additives

Examples of the electrolyte compound (e-1) include a combination of a metal iodide such as lithium iodide, sodium iodide, potassium iodide, cesium iodide or calcium iodide with iodine, a combination of a quaternary ammonium iodide such as tetraalkylammonium iodide, pyridium iodide or imidazolium iodide with iodine, a combination of a metal bromide such as lithium bromide, sodium bromide, potassium bromide, cesium bromide or calcium bromide with bromine, a combination of a quaternary ammonium bromide such as tetraalykylammonium bromide or pyridinium bromide with bromine, metal complexes such as ferrocyanic acid salt-ferricyanic acid salt or ferrocene-ferricynium ion, sulfur compounds such as sodium polysulfide and alkylthiolalkyldisulfide, a viologen dye, hydroquinone-quinone and a combination of a nitroxide radical such as 2,2,6,6-tetramethyl-1-piperidinyloxy (TEMPO) and oxoammonium salt. It is possible to prepare electrolyte compounds (e-1) by partially converting nitroxide radical into the oxoammonium salt in situ by adding oxidizing agent (e.g. NOBF₄).

The above electrolyte compounds (e-1) may be used solely or in the form of a mixture. As an electrolyte compound (e-1), there may be used a molten salt that is in a molten state at room temperature. When such a molten salt is used, particularly, it is not necessary to use a solvent.

The electrolyte compound (e-1) concentration in the electrolyte solution is preferably 0.05 to 20M, more preferably 0.1 to 15M.

For instance, the solvent (e-2) is nitrile solvents such as acetonitrile, methoxy acetonitrile, methoxy propionitrile, propionitrile and benzonitrile, carbonate solvents such as ethylene carbonate, propylene carbonate and diethyl carbonate, alcohol solvents such as methanol, ethanol, isopropyl alcohol, t-butyl alcohol, ethylene glycol and propylene glycol, ether solvents such as dioxane, diethyl ether, dimethoxyethane, tetrahydrofuran, dioxolane, t-butyl methyl ether, ethylene glycol dialkyl ether, propylene glycol monomethyl ether acetate and propylene glycol methyl ether, water, ketone solvents such as acetone, amide solvents such as N,N-dimethylformamide, N,N-dimethylacetamide and N-methylpyrrolidone, heterocyclic compounds such as 3-methyl-2-oxazolidinone, dimethyl sulfoxide, sulfolane and γ-butyrolactone, halogenated hydrocarbon solvents such as dichloromethane, chloroform, dichloroethane, trichloroethane, trichloroethylene, chlorobenzene, o-dichlorobenzene, 1-chloronaphthalene, bromoform, bromobenzene, methyl iodide, iodobenzene and fluorobenzene and hydrocarbon solvents such as benzene, toluene, o-xylene, m-xylene, p-xylene, ethylbenzene, cumene, n-pentane, n-hexane, n-octane, cyclohexane, methylcyclohexane, 1,5-hexadiene and cyclohexadiene or combinations of the above mentioned solvents and the ionic liquid is a quaternary imidazolium salt, a quaternary pyridinium salt, a quarternary ammonium salt or combinations thereof, preferably the anion of the salt is BF₄⁻, PF₆⁻, F(HF)₂⁻, F(HF)₃⁻, bis(trifluoromethanesulfonyl)imide [(CF₃SO₂)₂N⁻], N(CN)₂⁻, C(CN)₃⁻, B(CN)₄⁻, SCN⁻, SeCN⁻, I⁻, IO₃⁻ or combinations thereof.

For example, a photoelectric conversion device comprises a solvent (e.g. without an ionic liquid). For instance, a photoelectric conversion device comprises an ionic liquid (e.g. without a solvent).

Examples of further additives (e-3) are lithium salts (especially 0.05 to 2.0M, preferably 0.1 to 0.7M) (e.g. LiClO₄, LiSO₃CF₃ or Li(CF₃SO₂)N); pyridines (especially 0.005 to 2.0M, preferably 0.02 to 0.7M) (e.g. pyridine, tert-butylpyridine or polyvinylpyridine), gelling agents (especially 0.1 to 50 wt. %, preferably 1.0 to 10 wt. % based on the weight of the component e) (e.g. polyvinylidene fluoride, polyvinylidene fluoride-hexafluoropropylene copolymer, polyethylene oxide derivatives, polyacrylonitrile derivatives or amino acid derivatives), nano particles (especially 0.1 to 50 wt. %, preferably 1.0 to 10 wt. % based on the weight of the component e) (e.g. conductive nano particles, in particular single-wall carbon nanotubes, multi-wall carboncarbon nanotubes or combinations thereof, carbon fibers, carbon black, polyaniline-carbon black composite TiO₂, SiO₂ or SnO₂); and combinations thereof.

In the present invention, an inorganic solid compound such as copper iodide, copper thiocyanide or the like, an organic hole-transporting material or an electron-transporting material can be used in place of the electrolyte layer (e).

The instant electrode layer, photoelectric conversion devices and DSC can be prepared as outlined in U.S. Pat. No. 4,927,721, U.S. Pat. No. 5,084,365, U.S. Pat. No. 5,350,644 and U.S. Pat. No. 5,525,440 or in analogy thereto.

The present invention also pertains to a dye sensitized solar cell comprising a photoelectric conversion device as described herein.

The present invention also pertains to the use of a compound of formula (I) as defined herein as a dye in a dye sensitized solar cell.

The present invention further pertains to a compound of formula (I) as defined herein,

with the proviso that R₂ and R₁₅ and/or R₂ and R₁₆ do not form together an unsubstituted or substituted aliphatic 5-, 6- or 7-membered ring.

The compounds of formula (I) can be prepared according to methods known in the art.

Compounds of formula (I) can be prepared by condensation of the corresponding pyridinium salt and ketone as described below:

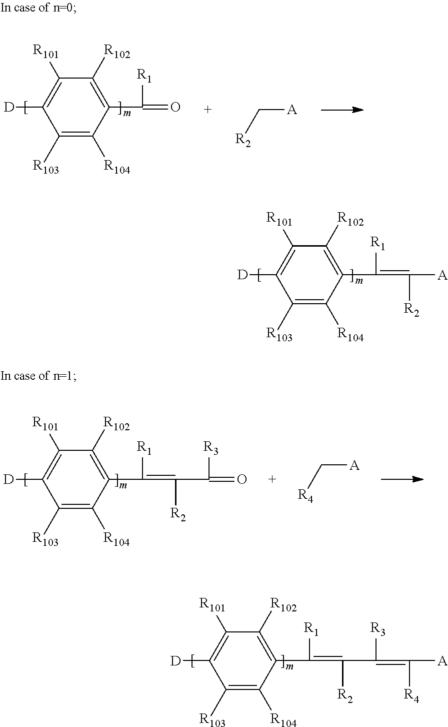

Compounds with n being 2-5 can be prepared accordingly.

For instance, carbonyl compounds can be prepared from corresponding amino phenyl derivatives via vilsmeyer reaction (see for instance, Tetrahedron letters, 46, 3913-3916, 2005), or reacting with dichloromethyl methyl ether in the presence of TiCl4 (see for instance, J. Org. Chem, 57(25), 6847-6852, 1992), giving desired carbonyl intermediates.

For example, biphenyl carbonyl intermediates (m=1) can be obtained via Suzuki coupling reactions below (see for instance; Bioorganic & Medicinal Chemistry, 16(16), 7715-7727, 2008);

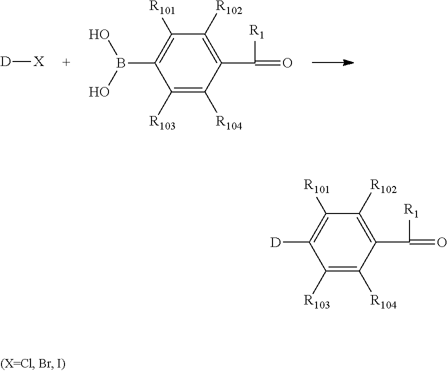

For instance, the reaction conditions of the condensation of the quaternary salts with carbonyl compounds are reflux in ethanol in the presence of piperidine or pyrrolidine (see for instance, J. Chem. Soc. 1961, 5074, Dyes & Pigments 2003, 58, 227), or heating in acetic anhydride (see for instance, Indian J. Chem. 1968, 6, 235.), or heating in acetic acid in a presence of ammonium acetate.

Before condensation, the groups G and G′ may be protected. Then after the condensation reaction, the protection group can be removed. A group G or G′ comprising COOH or COO⁻Z⁺ can be protected by, for example, t-butyl group. Then after condensation reactions, the COO-t-butyl group can be converted into COOH or COO⁻Z⁺. For instance, carboxylic acids are reacted with t-butanol in DMF in the presence of N,N′-carboxyldiimidazole and DBU (See for instance, Synthesis, communications, 1982, 833-834)

Or compounds of formula (I) can be prepared by condensation of the corresponding pyridine derivatives with carbonyl compounds, followed by quaternization to the corresponding pyridinium.

For instance, the starting materials are partly items of commerce or can be obtained according to methods known in the art.

When a denotation (e.g. D, G′, R₃-R₂₈) occurs more than once (e.g. twice) in a compound, this denotation may be different groups or the same group unless otherwise stated.

It is to be understood that alkyl and alkylene interrupted by O, S, C(═O), NR₁₄ or combinations comprises at least 2 carbon atoms and in case of combinations comprises at least 3 carbon atoms.

In the definitions the term alkyl comprises within the given limits of carbon atoms, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, 2-methylheptyl, 1,1,3,3-tetra-methylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl or dodecyl.

Examples of alkenyl are within the given limits of carbon atoms vinyl, allyl, 1-methylethenyl, and the branched and unbranched isomers of butenyl, pentenyl, hexenyl, heptenyl, octenyl, nonenyl, decenyl, undecenyl and dodecenyl. The term alkenyl also comprises residues with more than one double bond that may be conjugated or non-conjugated, for example may comprise one double bond.

Examples of alkynyl are within the given limits of carbon atoms ethynyl, propargyl, 1-methylethynyl, and the branched and unbranched isomers of butynyl, pentynyl, hexynyl, heptynyl, octynyl, nonynyl, decynyl, undecynyl and dodecynyl. The term alkynyl also comprises residues with more than one triple bond and residues with a triple bond and a double bond, all of which may be conjugated or non-conjugated. For instance, alkynyl comprises one triple bond.

Aryl is for example phenyl, biphenyl, naphthalinyl, anthracenyl, phenanthrenyl or pyrenyl, in particular phenyl or pyrenyl, especially phenyl.

Aryl/arylene can be (further) substituted by . . . is to be understood to include the aryl of aralkyl, aralkenyl, aralkynyl, aralkylene, aralkenylene and aralkynylene.

Heteroaryl may comprise one or more (e.g. 1-4, in particular 1-3, especially 1-2, such as 1 heteroatom preferably selected from the group consisting of O, S and N, especially S and N, in particular N). Examples of heteroaryl are thiophenyl, phenyl thiophenyl, diphenyl thiophenyl, triphenyl thiophenyl, bithiophenyl, terthiophenyl, tetrathiophenyl, furanyl, bifuranyl, terfuranyl, pyrrolyl, carbazolyl, phenyl carbazolyl, diphenyl carbazolyl, triphenyl carbazolyl, tetraphenyl carbazolyl, terphenyl carbazolyl, indolyl, piperidinyl, 9H-purinyl, pteridinyl, chinolinyl, isochinyl, acridinyl, phenazinyl,

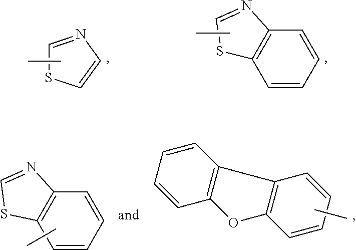

preferred examples are thiophenyl, phenyl thiophenyl, diphenyl thiophenyl, triphenyl thiophenyl, carbazolyl, phenyl carbazolyl, diphenyl carbazolyl, triphenyl carbazolyl, tetraphenyl carbazolyl, terphenyl carbazolyl,

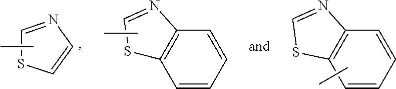

more preferred examples are thiophenyl, triphenyl thiophenyl, carbazolyl, phenyl carbazolyl,

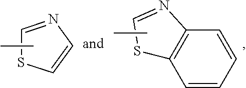

particularly preferred examples are terphenyl carbazolyl or phenyl carbazolyl.

Methyl (fluoren-9-ylidene) is for instance

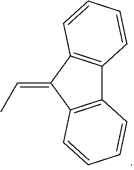

Aralkyl is for instance benzyl or α,α-dimethylbenzyl, especially benzyl.

Aralkenyl includes, for instance,

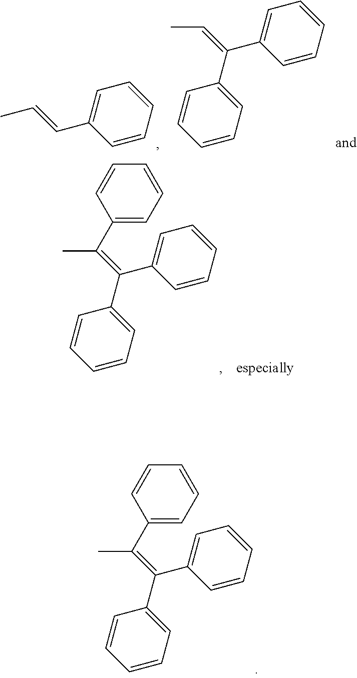

An example of aralkynyl is 2-phenylethynyl.

Some examples of cycloalkyl are

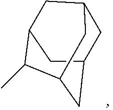

cyclobutyl, cyclopentyl, cyclohexyl, cycloheptyl, cyclooctyl, methylcyclopentyl, dimethylcyclopentyl, methylcyclohexyl and dimethylcyclohexyl, for instance cyclohexyl.

Some examples of cycloalkenyl are cyclopentenyl, cyclohexenyl, methylcyclopentenyl, dimethylcyclopentenyl and methylcyclohexenyl. Cycloalkenyl may comprise more than one double bond that may be conjugated or non-conjugated, for example may comprise one double bond.

Some examples of cycloalkynyl are cyclohexynyl and methylcyclohexenyl.

The term halogen may comprise fluorine, chlorine, bromine and iodine; for example halogen is fluorine.

In the definitions the term alkylene comprises within the given limits of carbon atoms, for example methylene, ethylene, propylene, isopropylene, n-butylene, sec-butylene, isobutylene, tert-butylene, 2-ethylbutylene, n-pentylene, isopentylene, 1-methylpentylene, 1,3-dimethylbutylene, n-hexylene, 1-methylhexylene, n-heptylene, 2-methylheptylene, 1,1,3,3-tetramethylbutylene, 1-methylheptylene, 3-methylheptylene, n-octylene, 2-ethylhexylene, 1,1,3-trimethylhexylene, 1,1,3,3-tetramethylpentylene, nonylene, decylene, undecylene, 1-methylundecylene or dodecylene.

Examples of alkenylene are within the given limits of carbon atoms vinylene, allylene, 1-methylethenylene, and the branched and unbranched isomers of butenylene, pentenylene, hexenylene, heptenylene, octenylene, nonenylene, decenylene, undecenylene and dodecenylene. The term alkenylene also comprises residues with more than one double bond that may be conjugated or non-conjugated, for example may comprise one double bond.

Examples of alkynylene are within the given limits of carbon atoms ethynylene, propargylene, 1-methylethynylene, and the branched and unbranched isomers of butynylene, pentynylene, hexynylene, heptynylene, octynylene, nonynylene, decynylene, undecynylene and dodecynylene. The term alkynylene also comprises residues with more than one triple bond and residues with a triple bond and a double bond, all of which may be conjugated or non-conjugated. For instance, alkynylene comprises one triple bond.

Arylene is for example phenylene, biphenylene, naphthalinylene, anthracenylene, phenanthrenylene or pyrenylene, in particular phenylene.

Heteroarylene may comprise one or more (e.g. 1-4, in particular 1-3, especially 1-2, such as 1 heteroatom preferably selected from the group consisting of O, S and N, especially S and N, in particular N). Examples of heteroarylene are thiophenylene, bithiophenylene, terthiophenylene, tetrathiophenylene, furanylene, bifuranylene, terfuranylene, pyrrolylene, carbazolylene, indolylene, piperidinylene, 9H-purinylene, pteridinylene, chinolinylene, isochinylene, acridinylene, phenazinylene and

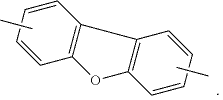

Aralkylene is for instance phenylmethylene, phenylethylene or phenyldimethylmethylene.

Aralkenyl includes, for instance, phenylethenylene.

An example of aralkynylene is phenylethynylene.

Some examples of cycloalkylene are cyclobutylene, cyclopentylene, cyclohexylene, cycloheptylene, cyclooctylene, methylcyclopentylene, dimethylcyclopentylene, methylcyclohexylene and dimethylcyclohexylene, for instance cyclohexylene.

Some examples of cycloalkenylene are cyclopentenylene, cyclohexenylene, methylcyclopentenylene, dimethylcyclopentenylene and methylcyclohexenylene. Cycloalkenylene may comprise more than one double bond that may be conjugated or non-conjugated, for example may comprise one double bond.

Some examples of cycloalkynylene are cyclohexynylene and methylcyclohexynylene.

Examples of heteroaralkylene are thiophenylmethylene, thiophenylethylene, bithiophenylmethylene, bithiophenylethylene, furanylmethylene, furanylethylene, bifuranylmethylene bifuranylethylene, pyrrolylmethylene, pyrrolylethylene, indolylmethylene, indolylethylene, chinolinylmethylene, chinolinylethylene, isochinolinylmethylene and isochinolinylethylene.

Examples of heteroaralkenylene are thiophenylethenylene, bithiophenylethenylene, furanylethenylene, bifuranylethenylene, pyrrolylethenylene, indolylethenylene, chinolinylethenylene and isochinolinylethenylene.

Examples of heteroaralkynylene are thiophenylethynylene, bithiophenylethynylene, furanylethynylene, bifuranylethynylene, pyrrolylethynylene, indolylethynylene, chinolinylethynylene and isochinolinylethynylene.

For instance, fluorinated alkyl is an alkyl substituted by one or more fluorine. Examples of fluorinated alkyl are —CF₃, —CF₂CF₃, —CF₂CF₂CF₃, —CF(CF₃)₂, —(CF₂)₃CF₃, —(CF₂)CF₂H and —C(CF₃)₃, in particular —CF₃. Preferably, fluorinated alkyl is perfluorinated.

The preferences outlined herein apply to all aspects of the invention.

Ratio and % are weight ratio and weight-% unless otherwise stated.

### ABBREVIATIONS

DSC dye sensitized solar cell

### PREPARATION EXAMPLES

### Example 1

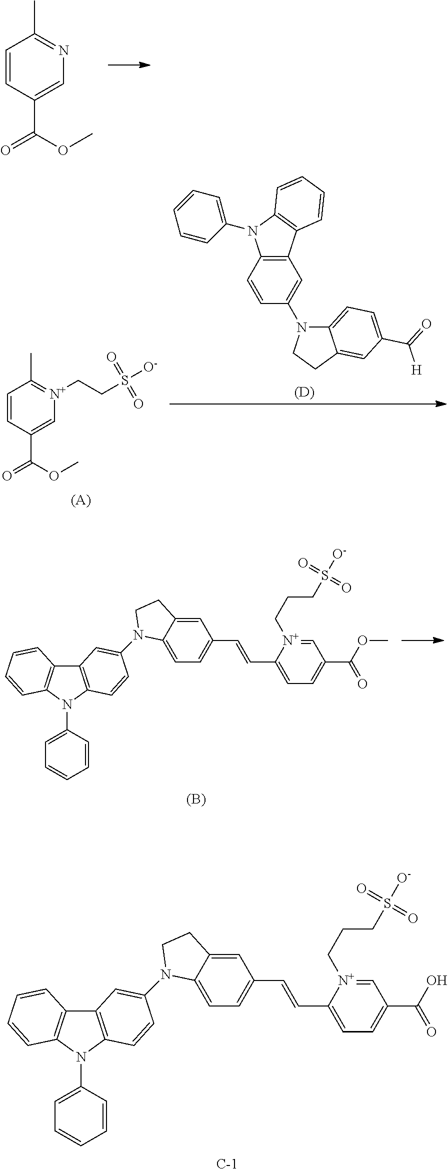

### Preparation of (A);

0.5 g (3.3 mmol) of Methyl-6-methylnicotionate and 1.6 g (13.2 mmol) of Propane sultone are dissolved in 8 ml of acetonitrile and the solution is stirred at 90° C. for 15 hours. After cooling down to room temperature, the reaction mixture is concentrated under reduced pressure. The crude product is purified by column chromatography and 1.06 g of yellow solid (A) is obtained (yield 80%).

### Preparation of (B);

150 mg (0.37 mmol) of (A), 143 mg (0.37 mmol) of (D), 0.32 mg (0.0037 mmol) of piperidine and 0.22 mg (0.0037 mmol) of acetic acid are dissolved in 7 ml of toluene and the solution is stirred at 110° C. for 15 hours. After cooling down to room temperature, the reaction mixture is concentrated under reduced pressure. The crude product is purified by column chromatography and 132 mg of wine red solid (B) is obtained (yield 55%).

### Preparation of C-1;

To 5 ml of Pyridine 132 mg (0.21 mmol) of pyridinium salt (B) and 112 mg (0.84 mmol) of Lithium iodide are added. The mixture is stirred at 110° C. for 18 hours. After cooling down to room temperature, the reaction mixture is concentrated under reduced pressure. The crude product is purified by column chromatography and 108 mg of dark purple solid C-1 is obtained (yield 82%).

¹H NMR (400 MHz, CDCl₃): δ (ppm)=8.95 (s, 1H), 8.25-8.5 (m, 3H), 8.18 (s, 1H), 7.88-7.95 (d, 2H), 7.22-7.70 (m, 12H), 4.80-4.90 (t, 2H), 4.1-4.2 (t, 2H), 3.15-3.25 (m, 2H), 2.5-2.6 (m, 2H), 2.0-2.2 (m, 2H)

### Example 2

C-2 is prepared in analogy to Example 1 procedures except using the corresponding aldehyde in place of (D).

¹H NMR (400 MHz, CDCl₃): δ (ppm)=8.91 (s, 1H), 8.44-8.46 (d, 1H), 8.16-8.18 (d, 1H), 7.95-7.99 (d, 1H), 7.86-7.88 (d, 1H), 7.35-7.39 (d, 1H), 6.29-6.31 (d, 1H), 6.14 (s, 1H), 4.75-4.79 (m, 2H), 4.07-4.1 (m, 2H), 3.33-3.37 (m, 2H), 2.5-2.7 (m, 2H), 2.05-2.15 (m, 2H), 1.75-1.85 (m, 2H), 1.2-1.6 (m, 24H), 0.8-0.9 (m, 9H)

### Example 3

C-6 is prepared in analogy to Example 1 except using the corresponding pyridinium salt in place of (A).

¹H NMR (400 MHz, CDCl₃): δ (ppm)=8.91 (s, 1H), 8.4-8.5 (d, 1H), 8.3-8.4 (d, 1H), 7.8-7.9 (d, 1H), 7.7-7.8 (d, 2H), 7.4-7.5 (d, 1H), 6.65-6.75 (d, 2H), 4.8-4.9 (m, 2H), 3.0 (s, 6H), 2.5-2.6 (m, 2H), 2.0-2.2 (m, 2H)

### Application Examples

### Example 4

Titanium oxide paste (PST-18NR, supplied by Catalysts&Chemicals Ind. Co., Ltd.) is applied onto a slide glass substrate by screen printing method. After being dried for 5 minutes at 120° C., a substrate having a thickness of 1.5 μm layer of TiO₂ is obtained by applying heat treatment in air at 450° C. for 30 minutes and 500° C. for 30 minutes.

0.02 g of a dye (C-1) is dissolved in 25 ml of a mixture solution of acetonitrile+t-butyl alcohol+ethanol (1:2:1). The above-prepared substrate is immersed in the solution at room temperature for 2 hours so as to adsorb the dye.

The dye C-1 amount on TiO₂ is estimated from the transmittance spectrum of the above substrate measured with UV-Vis spectrometer (U-3300, Hitachi High-Tech Fielding Corporation). The above substrate is then immersed into 2 ml of gamma-butyrolactone (GBL) in the dark for three days, and again the dye C-1 amount remaining on TiO₂ is estimated from the transmittance spectrum. The dye's adsorptivity is estimated from the ratio of dye remaining on TiO₂ after immersion. After 3 days immersion in GBL, 97% of compound C-1 remains on TiO₂ electrode.

### Example 5 to 8

The adsorptivity of dye C-1 is estimated in the same manner as described in Example 4 except that the acetnitrile is replaced with propylene carbonate (PC), acetnitrile (MeCN), 1-methyl-3-propyl imidazolium iodide (MPImI), 1-hexyl-3-methyl imidazolium iodide (HMImI). The data is summarized in Table 1.

### Example 9-18

The adsorptivities of compounds C-2 and C-6 are estimated in the same manner as described in Example 4-8 except that the dye is replaced with compound C-2 or C-6. The data is summarized in Table 1.

### Comparative Example 19-23

The adsorptivity of R-1 is estimated in the same manner as described in Example 4-8 except that the dye is replaced with R-1 shown below. The data is summarized in Table 1.

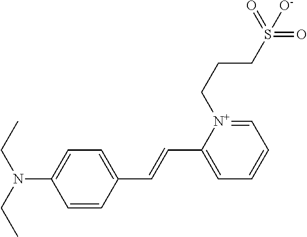

prior art compound R-1 (dye 2 of CN-C-1253436)

### Example 24-28

The adsorptivities of compound C-39 are estimated in the same manner as described in Example 4-8 except that the dye is replaced with compound C-39. The data is summarized in Table 1.

TABLE 1

| | Dye Remained/% | | | | |
|---|---|---|---|---|---|
| | Example | | | | Reference |
| Solvent | C-1 | C-2 | C-6 | C-39 | R-1 |
| GBL | 70 (Ex-4) | 49 (Ex-9) | 60 (Ex-14) | 100 (Ex-24) | 32 (Ex-19) |
| PC | 88 (Ex-5) | 75 (Ex-10) | 78 (Ex-15) | 97 (Ex-25) | 35 (Ex-20) |
| MeCN | 97 (Ex-6) | 100 (Ex-11) | 89 (Ex-16) | 100 (Ex-26) | 0 (Ex-21) |
| MPImI | 94 (Ex-7) | 99 (Ex-12) | 67 (Ex-17) | 99 (Ex-27) | 40 (Ex-22) |
| HMImI | 93 (Ex-8) | 98 (Ex-13) | 73 (Ex-18) | 81 (Ex-28) | 39 (Ex-23) |

As shown in the table 1, the dyes of present invention show better adsorptivity than the prior art compound (R-1) in practical solvents used for DSC device, providing stable DSC performance.

### Further Preparation Examples

### Example 29

C-4 is prepared in analogy to Example 1 procedures except using the precursor (A′) and the corresponding aldehyde in place of (D).

¹H NMR (400 MHz, DMSO-d6): δ (ppm)=8.95 (s, 1H), 8.25-8.5 (m, 3H), 8.18 (s, 1H), 7.88-7.95 (d, 2H), 7.22-7.70 (m, 12H), 4.80-4.90 (t, 2H), 4.1-4.2 (t, 2H), 3.15-3.25 (m, 2H), 2.5-2.6 (m, 2H), 2.0-2.2 (m, 2H)

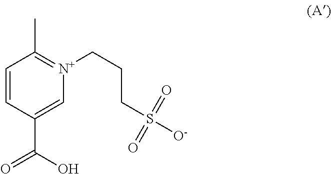

### Example 30

C-39 is prepared in analogy to Example 29 procedures except using the corresponding aldehyde in place of (D).

¹H NMR (400 MHz, DMSO-d6): δ (ppm)=8.9 (s, 1H), 7.95-8.5 (m, 5H), 7.2-7.7 (m, 11H), 6.5 (s, 1H), 4.75-4.90 (t, 2H), 4.1-4.3 (t, 2H), 3.90-4.05 (t, 2H), 3.05-3.15 (t, 2H), 2.5-2.6 (m, 2H), 2.05-2.15 (m, 2H), 1.65-1.8 (m, 2H), 1.1-1.4 (m, 12H), 0.7-0.85 (m, 3H), 4.80-4.90 (t, 2H), 4.1-4.2 (t, 2H), 3.15-3.25 (m, 2H), 2.5-2.6 (m, 2H), 2.0-2.2 (m, 2H)

### Example 31

C-40 is prepared in analogy to Example 29 procedures except using the corresponding aldehyde in place of (D).

¹H NMR (400 MHz, DMSO-d6): δ (ppm)=8.96 (s, 1H), 8.66 (s, 1H), 8.50 (d, 1H), 8.21-8.34 (m, 4H), 7.92-8.02 (m, 2H), 7.63-7.70 (m, 9H), 7.46-7.56 (m, 3H), 7.38-7.46 (m, 4H), 7.34 (d, 1H), 7.23 (t, 1H), 7.18 (d, 1H), 6.50 (s, 1H), 6.39 (d, 1H), 4.78-4.83 (m, 2H), 3.63 (s, 3H), 2.07-2.15 (m, 2H); 2H signal is hidden by H2O signal around 3.3 ppm.

### Example 32

C-41 is prepared in analogy to Example 29 procedures except using the corresponding aldehyde in place of (D).

¹H NMR (400 MHz, DMSO-d6): δ (ppm)=8.98 (s, 1H), 8.37-8.54 (m, 3H), 8.29 (d, 1H), 8.18 (s, 1H), 7.71 (d, 4H), 7.40-7.58 (m, 7H), 7.29 (t, 2H), 7.19 (t, 1H), 6.96 (t, 1H), 6.74 (d, 1H), 6.69 (d, 1H), 6.20 (d, 1H), 4.88-4.96 (m, 2H), 4.41 (t, 1H), 3.40-3.62 (m, 2H), 2.57-2.63 (m, 2H), 2.12-2.21 (m, 2H)

### Example 33

C-42 is prepared in analogy to Example 29 procedures except using the corresponding aldehyde in place of (D).

¹H NMR (400 MHz, DMSO-d6): δ (ppm)=8.98 (s, 1H), 8.37-8.54 (m, 3H), 8.29 (d, 1H), 8.18 (s, 1H), 7.71 (d, 4H), 7.40-7.58 (m, 7H), 7.29 (t, 2H), 7.19 (t, 1H), 6.96 (t, 1H), 6.74 (d, 1H), 6.69 (d, 1H), 6.20 (d, 1H), 4.88-4.96 (m, 2H), 4.41 (t, 1H), 2.57-2.63 (m, 2H), 2.12-2.21 (m, 2H), 1.7-1.9 (m, 2H), 1.2-1.5 (m, 6H), 3H signal is hidden by H2O signal around 3.3 ppm.

### Example 34

C-43 is prepared in analogy to Example 29 procedures except using the corresponding aldehyde in place of (D)

¹H NMR (400 MHz, DMSO-d6): δ (ppm)=8.98 (s, 1H), 8.7-8.90 (dd, 2H), 8.0 (s, 1H), 7.9-7.95 (d, 1H), 6.95-7.6 (m, 17H), 6.85-6.95 (d, 1H), 4.87-4.98 (m, 2H), 4.23-4.32 (m, 1H), 2.50-2.60 (m, 2H), 2.10-2.20 (m, 2H), 0.98-2.03 (m, 16H)

### Example 35

C-44 is prepared in analogy to Example 1 procedures except using the precursor (A″) and the corresponding aldehyde in place of (D).

¹H NMR (400 MHz, DMSO-d6): δ (ppm)=9.05-9.15 (bs, 1H), 8.4-8.5 (d, 1H), 8.18-8.26 (d, 1H), 7.82-7.92 (d, 1H), 7.52-7.60 (d, 1H), 7.24-7.34 (d, 1H), 6.35-6.45 (d, 1H), 6.2 (s, 1H), 4.05-4.15 (t, 2H), 1.9-2.1 (m, 2H), 1.75-1.9 (m, 4H), 1.2-1.7 (m, 14H), 1.1-1.2 (t, 6H), 0.8-0.95 (t, 3H) 2H signal is hidden by H2O signal around 3.3 ppm.

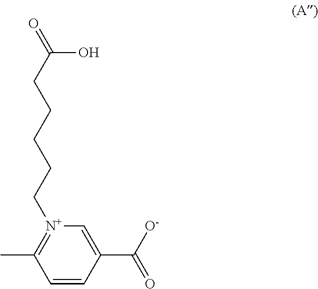

### Example 36

C-45 is prepared in analogy to Example 1 procedures except using the precursor (A′″) and the corresponding aldehyde in place of (D).

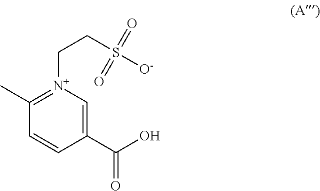

¹H NMR (400 MHz, DMSO-d6): δ (ppm)=8.92 (s, 1H), 8.47 (d, 1H), 8.28 (t, 2H), 8.18 (s, 1H), 7.84 (d, 1H), 7.76 (s, 1H), 7.61-7.70 (m, 4H), 7.34-7.54 (m, 7H), 7.25 (t, 1H), 6.95 (d, 1H), 4.81-4.88 (m, 2H), 4.16 (t, 2H), 3.23 (t, 2H), 3.05 (t, 2H)

What is claimed:

1. An electrode layer comprising a porous film made of oxide semiconductor fine particles sensitized with a dye of formula (I), 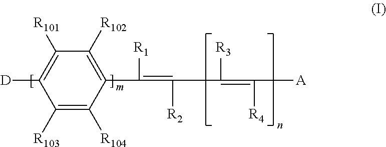
wherein
m is 0 or 1;
n is 0, 1, 2, 3, 4 or 5;
R₁, R₂, R₃ and R₄ are independently H, —S(═O)₂OR₇, —S(═O)₂R₇, —S(═O)R₇, —S(═O)OR₇, fluorinated C₁-C₈alkyl, a group of formula (II) 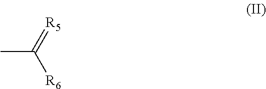
or unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof; or R₁ and R₂, or R₃ and R₄ form together an unsubstituted or substituted aliphatic 5-, 6- or 7-membered ring;
or if m is 0, R₁ and R₁₅, R₁ and R₁₆, R₂ and R₁₅ or R₂ and R₁₆ can form together an unsubstituted or substituted aliphatic 5-, 6- or 7-membered ring;
or if m is 0, R₂ and R₁₅ can form together an unsubstituted or substituted heteroaromatic 5-, 6- or 7-membered ring;
or a substituent R₃₀-R₃₃ of A ortho to the 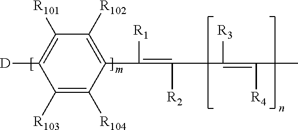
group and either if n is 0, R₂ or if n is 1, R₄ can form together an unsubstituted or substituted aliphatic 5-, 6- or 7-membered ring;
or R₁ is D;
with the proviso that A is a group of formula (VI) or (VII) if at least one of R₁-R₄ is fluorinated C₁-C₈alkyl, —S(═O)₂OR₇, —S(═O)₂R₇, —S(═O)R₇, —S(═O)OR₇ or a group of formula (II) 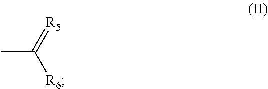
R₅ is NR₈, N—OR₈, N—NR₈R₉, O or S;
R₆ is CO—SR₇, CO—NR₇—NR₁₀R₁₁, CO—NR₇—OR₁₀, CO—O—CO—R₇, CO—NR₇—CO—R₁₀, CO—NR₇—CO—OR₁₀, CO—NR₇—CO—NR₁₀R₁₁, NR₇R₁₀, OR₇, SR₇, NR₇—NR₁₀R₁₁, NR₇—OR₁₀, O—CO—R₇, O—CO—OR₇, O—CO—NR₇R₁₀, NR₇—CO—R₁₀, NR₇—CO—OR₁₀, NR₇—CO—NR₁₀R₁₁, CO—R₇, CO—OR₇, CO—NR₇R₁₀, NR₁₂—C(═NR₁₃)R₇ or unsubstituted or substituted C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
R₈ and R₉ are independently H or unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
R₇, R₁₀ and R₁₁ are independently H or unsubstituted or substituted C₁-C₂₀alkyl, C₂-C₂₀alkenyl, C₂-C₂₀alkynyl, C₆-C₂₀aryl, C₃-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
or R₇ or R₁₀ together with that group of R₁-R₄ which is attached to the same double bond as the group of R₁-R₄ comprising said R₇ or R₁₀ forms an unsubstituted or substituted aliphatic 5-, 6- or 7-membered ring; or if R₇ or R₁₀ is part of R₂ with n being 0 or is part of R₄ with n being 1, it can form together with a substituent R₃₀-R₃₃ of A ortho to the 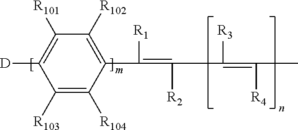
group an unsubstituted or substituted aliphatic 5-, 6- or 7-membered ring;
R₁₂ and R₁₃ form together an unsubstituted or substituted 5-, 6- or 7-membered ring;
R₁₄ is independently H or unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl;
D is independently a group of formula (III) or (IV) 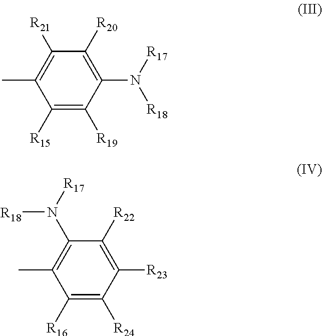
R₁₇ and R₁₈ are independently unsubstituted or substituted C₁-C₂₀alkyl, C₂-C₂₀alkenyl, C₂-C₂₀alkynyl, C₆-C₂₀aryl, C₄-C₃₆heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof; or R₁₇ and R₁₈, R₁₇ and R₂₂, R₁₇ and R₂₀ and/or R₁₈ and R₁₉ form together an unsubstituted or substituted 5-, 6- or 7-membered ring;
R₁₅, R₁₆, R₁₉, R₂₀, R₂₁, R₂₂, R₂₃ and R₂₄ are independently H, NR₂₅R₂₆, OR₂₅, SR₂₅, NR₂₅—NR₂₆R₂₇, NR₂₅—OR₂₆, O—CO—R₂₅, O—CO—OR₂₅, O—CO—NR₂₅R₂₆, NR₂₅—CO—R₂₆, NR₂₅—CO—OR₂₆, NR₂₅—CO—NR₂₆R₂₇, CO—R₂₅, CO—OR₂₅, CO—NR₂₅R₂₆, S—CO—R₂₅, CO—SR₂₅, CO—NR₂₅—NR₂₆R₂₇, CO—NR₂₅—OR₂₆, CO—O—CO—R₂₅, CO—O—CO—OR₂₅, CO—O—CO—NR₂₅R₂₆, CO—NR₂₅—CO—R₂₆, CO—NR₂₅—CO—OR₂₆, or unsubstituted or substituted C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₅-C₂₀aralkenyl, C₅-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
R₂₅, R₂₆ and R₂₇ are independently H or unsubstituted or substituted C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₅-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
A is a group of formula (V), (VI) or (VII) 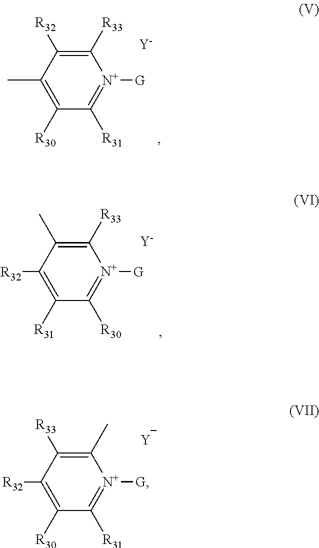
Y⁻ is an inorganic or organic anion, wherein the inorganic anion is Cl⁻, Br⁻, I⁻, SCN⁻, BF₄⁻, PF₆⁻, ClO₄⁻, SbF₆⁻, or AsF₆⁻; and the organic anion is carboxylate, sulphonate, sulphate, phosphate, phosphonate, —CO(C═O)O⁻ or —NR₁₄—S(═O)₂O⁻;
G and G′ are independently —R₂₈—COOH, —R₂₈—COO⁻Z⁺, —R₂₈—CO(C═O)OH, —R₂₈—CO(C═O)O⁻Z⁺, —R₂₈—S(═O)₂OH, —R₂₈—S(═O)₂O⁻Z⁺, —R₂₈—O—S(═O)₂OH, —R₂₈—O—S(═O)₂O⁻Z⁺, —R₂₈—P(═O)(OH)₂, —R₂₈—P(═O)(O⁻Z⁺)₂, —R₂₈—P(═O)(OH)(O⁻Z⁺), —R₂₈—O—P(═O)(OH)₂, —R₂₈—O—P(═O)(O⁻Z⁺)₂, —R₂₈—O—P(═O)(OH)(O⁻Z⁺), —R₂₈—CO—NH—OH, —R₂₈—S(═O)₂NH—OH, —R₂₈—NR₁₄—S(═O)₂OH or —R₂₈—NR₁₄—S(═O)₂O⁻Z⁺;
Z⁺ is N(R₁₄)₄⁺, Li⁺, Na⁺ or K⁺ or is an ammonium cation which is part of a compound of formula (I) as part of group A;
R₂₈ is a direct bond or unsubstituted or substituted C₁-C₂₀alkylene, C₂-C₂₀alkenylene, C₂-C₂₀alkynylene, C₆-C₂₀arylene, C₄-C₉heteroarylene, C₇-C₁₁aralkylene, C₅-C₁₁aralkenylene, C₈-C₁₁aralkynylene, C₆-C₁₁heteroaralkylene, C₇-C₁₁heteroaralkenylene, C₇-C₁₁heteroaralkynylene, C₅-C₆cycloalkylene, C₁-C₂₀alkylene-C₆-C₂₀arylene, C₁-C₂₀alkylene-C₄-C₉heteroarylene, C₁-C₂₀alkylene-C₆-C₂₀arylene-C₁-C₂₀alkylene, C₁-C₂₀alkylene-C₄-C₉heteroarylene-C₁-C₂₀alkylene, —O—C₁-C₂₀alkylene, —O—C₂-C₂₀alkenylene, —O—C₂-C₂₀alkynylene, —O—C₆-C₂₀arylene, —O—C₄-C₉heteroarylene, —O—C₇-C₁₁aralkylene, —O—C₈-C₁₁aralkenylene, —O—C₈-C₁₁aralkynylene, —O—C₆-C₁₁heteroaralkylene, —O—C₇-C₁₁heteroaralkenylene, —O—C₇-C₁₁heteroaralkynylene, —O—C₅-C₆cycloalkylene, —O—C₁-C₂₀alkylene-C₆-C₂₀arylene, —O—C₁-C₂₀alkylene-C₄-C₉heteroarylene, —O—C₁-C₂₀alkylene-C₆-C₂₀arylene-C₁-C₂₀alkylene or —O—C₁-C₂₀alkylene-C₄-C₉heteroarylene-C₁-C₂₀alkylene;
R₃₀, R₃₁, R₃₂ and R₃₃ are independently G′, H, halogen, unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof; or 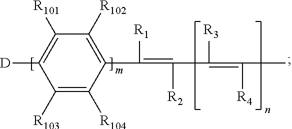
or two vicinal groups of R₃₀-R₃₃ form together an unsubstituted or substituted aliphatic 5-, 6- or 7-membered ring;
with the proviso that at least one of R₃₀-R₃₃ is G′;
R₁₀₁, R₁₀₂, R₁₀₃ and R₁₀₄ are independently H, halogen, unsubstituted or substituted C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof;
or R₁₀₂ or R₁₀₄ is D;
or R₁₀₁ and R₁₀₂ and/or R₁₀₃ and R₁₀₄ form together an unsubstituted or substituted aliphatic 5-, 6- or 7-membered ring;
or R₁₀₁ and R₁₅ or R₁₀₁ and R₁₆ form together an unsubstituted or substituted 5-, 6- or 7-membered ring.

2. An electrode layer according to claim 1, wherein
m is 0 or 1;
n is 0 or 1;
R₁, R₂, R₃ and R₄ are independently H, —S(═O)₂OR₇, —S(═O)₂R₇, —S(═O)R₇, —S(═O)OR₇, fluorinated C₁-C₈alkyl, a group of formula (II), C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, fluorinated C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof;
or R₁ is D;
or if m is 0, R₁ and R₁₅ or R₁ and R₁₆ can form together ethylene, trimethylene, tetramethylene, methylene-O, O-methylene, ethylene-O, O-ethylene, trimethylene-O, O-trimethylene, methylene-NR₁₄, NR₁₄-methylene, ethylene-NR₁₄, NR₁₄-ethylene, trimethylene-NR₁₄ or NR₁₄-trimethylene thus forming an aliphatic 5-, 6- or 7-membered ring;
or if m is 0, R₂ and R₁₅ can form together —O—, —S—, —C(O)— or —NR₁₄—;
or if m is 0 and D is a group of formula (IV), R₁ and R₁₈ can form together with the N-atom R₁₇ and R₁₈ are attached to 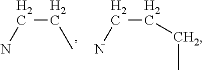
whereby in each of said groups one or more H-atom can be replaced by C₁-C₂₀alkyl, C₁-C₂₀alkylidene, phenyl or combinations thereof, in each of said groups two geminal H-atom can be replaced by C₁-C₂₀alkylidene, and/or in each of said groups two vicinal H-atoms can be replaced by benzo, trimethylene or tetramethylene;
with the proviso that A is a group of formula (VI) or (VII) if at least one of R₁-R₄ is fluorinated C₁-C₈alkyl, —S(═O)₂OR₇, —S(═O)₂R₇, —S(═O)R₇, —S(═O)OR₇ or a group of formula (II) 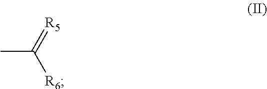
R₅ is NR₈, N—OR₈, N—NR₈R₉, O or S;
R₆ is NR₇R₁₀, OR₇, SR₇, NR₇—NR₁₀R₁₁, NR₇—OR₁₀, O—CO—R₇, O—CO—OR₇, O—CO—NR₇R₁₀, NR₇—CO—R₁₀, NR₇—CO—OR₁₀, NR₇—CO—NR₁₀R₁₁, CO—R₇, CO—OR₇, CO—NR₇R₁₀, C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₅-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, fluorinated C₁-C₂₀alkyl, C₂-C₂₀alkenyl, C₂-C₂₀alkynyl, C₇-C₂₀aralkyl, C₅-C₂₀aralkenyl, C₅-C₂₀aralkynyl, C₄-C₂₀cycloalkyl, C₅-C₂₀cycloalkenyl, C₆-C₂₀cycloalkynyl or combinations thereof;
R₈ and R₉ are independently H, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₅-C₂₀aralkenyl, C₅-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, fluorinated C₁-C₂₀alkyl, C₂-C₂₀alkenyl, C₂-C₂₀alkynyl, C₇-C₂₀aralkyl, C₈-C₂₀aralkenyl, C₅-C₂₀aralkynyl, C₄-C₂₀cycloalkyl, C₅-C₂₀cycloalkenyl, C₆-C₂₀cycloalkynyl or combinations thereof;
R₇, R₁₉ and R₁₁ are independently H, C₁-C₂₀alkyl, C₂-C₂₀alkenyl, C₂-C₂₀alkynyl, C₆-C₂₀aryl, C₃-C₂₀heteroaryl, C₇-C₂₀aralkyl, C₅-C₂₀aralkenyl, C₅-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, fluorinated C₁-C₂₀alkyl, fluorinated O—C₁-C₂₀alkyl, —CN, C₂-C₂₀alkenyl, C₂-C₂₀alkynyl, C₇-C₂₀aralkyl, C₅-C₂₀aralkenyl, C₅-C₂₀aralkynyl, C₄-C₂₀cycloalkyl, C₅-C₂₀cycloalkenyl, C₆-C₂₀cycloalkynyl or combinations thereof;
or R₇ or R₁₀ being part of R₂ forms together with R₁ a direct bond, methylene or ethylene thus forming an aliphatic 5-, 6- or 7-membered ring;
or R₇ or R₁₀ being part of R₄ forms together with R₃ a direct bond, methylene or ethylene thus forming an aliphatic 5-, 6- or 7-membered ring;
or if m is 0 and R₇ or R₁₀ is part of R₂, it can form together with R₁₅ or R₁₆ a direct bond or methylene thus forming an aliphatic 6- or 7-membered ring;
or if R₇ or R₁₀ is part of R₂ with n being 0 or is part of R₄ with n being 1, it can form together with a substituent R₃₀-R₃₃ of A ortho to the 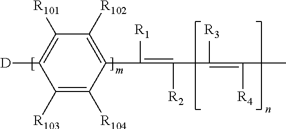
group a direct bond, methylene or ethylene thus forming an aliphatic 5-, 6- or 7-membered ring;
R₁₄, R₁₄′ are independently H, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl;
D is independently a group of formula (III) or (IV)
R₁₇ and R₁₈ are independently fluorenyl, C₁-C₂₀alkyl, C₂-C₂₀alkenyl, C₂-C₂₀alkynyl, C₆-C₂₀aryl, C₄-C₃₆heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the fluorenyl, alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by tetrahydrofuranyl, halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the fluorenyl, aryl and heteroaryl can be further substituted by maleic anhydridyl, maleimidyl, indenyl, C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof, whereby the maleic anhydridyl and maleimidyl are unsubstituted or substituted by C₁-C₂₀alkyl, C₆-C₂₀aryl, phenyl-NR₁₄R₁₄′ or combinations thereof;
or R₁₇ and R₁₈ form together with the N they are attached to piperidinyl, piperazinyl, morpholinyl, imidazolidinyl or pyrrollidinyl, each of which is unsubstituted or substituted by C₁-C₂₀alkyl, C₁-C₂₀alkylidene, benzo, trimethylene, tetramethylene or combinations thereof, which are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof;
or R₁₇ and R₂₂, R₁₇ and R₂₀ and/or R₁₈ and R₁₉ form together with the N-atom R₁₇ and R₁₈ are attached to 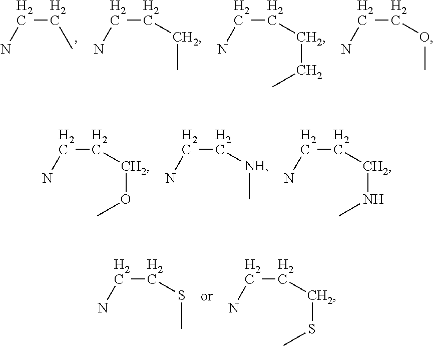
whereby in each of said groups one or more H-atom can be replaced by C₁-C₂₀alkyl, C₁-C₂₀alkylidene, phenyl, CO—OR₁₄, CONR₁₄R₁₄′ or combinations thereof, in each of said groups two geminal H-atom can be replaced by C₁-C₂₀alkylidene, and/or in each of said groups two vicinal H-atoms can be replaced by benzo, trimethylene or tetramethylene, whereby the benzo is unsubstituted or substituted by methyl (fluoren-9-ylidene);
R₁₅, R₁₆, R₁₉, R₂₀, R₂₁, R₂₂, R₂₃ and R₂₄ are independently H, NR₂₅R₂₆, OR₂₅, SR₂₅, NR₂₅—NR₂₆R₂₇, NR₂₅—OR₂₆, O—CO—R₂₅, O—CO—OR₂₅, O—CO—NR₂₅R₂₆, NR₂₅—CO—R₂₆, NR₂₅—CO—OR₂₆, NR₂₅—CO—NR₂₆R₂₇, CO—R₂₅, CO—OR₂₅, CO—NR₂₅R₂₆, CO—SR₂₅, CO—NR₂₅—NR₂₆R₂₇, CO—NR₂₅—OR₂₆, CO—O—CO—R₂₅, CO—O—CO—OR₂₅, CO—O—CO—NR₂₅R₂₆, CO—NR₂₅—CO—R₂₆, CO—NR₂₅—CO—OR₂₆, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof;
R₂₅, R₂₆ and R₂₇ are independently H, C₁-C₂₀alkyl, C₆-C₂₀aryl, C₂-C₂₀alkenyl, C₂-C₂₀alkynyl, C₆-C₂₀aryl, C₄-C₂₀heteroaryl, C₇-C₂₀aralkyl, C₅-C₂₀aralkenyl, C₅-C₂₀aralkynyl, C₄-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by pyridinium*Y⁻, maleic anhydridyl, maleimidyl, halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, C₂-C₂₀alkenyl, C₂-C₂₀alkynyl, C₇-C₂₀aralkyl, C₅-C₂₀aralkenyl, C₅-C₂₀aralkynyl, C₄-C₂₀cycloalkyl, C₅-C₂₀cycloalkenyl, C₆-C₂₀cycloalkynyl or combinations thereof, whereby the pyridinium, maleic anhydridyl, maleimidyl are unsubstituted or substituted by C₁-C₂₀alkyl, C₂-C₂₀alkenyl, C₂-C₂₀alkynyl, C₆-C₂₀aryl, C₆-C₂₀aryl-O—R₁₄, C₇-C₂₀aralkyl, C₅-C₂₀aralkenyl, C₅-C₂₀aralkynyl, C₄-C₂₀cycloalkyl, C₅-C₂₀cycloalkenyl, C₆-C₂₀cycloalkynyl, S—R₁₄, O—R₁₄, CO—OR₁₄, O—CO—R₁₄, NR₁₄R₁₄′, CONR₁₄R₁₄′, NR₁₄—CO—R₁₄′ or combinations thereof;
A is a group of formula (V), (VI) or (VII),
Y⁻ is Cl⁻, Br⁻, I⁻, SCN⁻, BF₄⁻, PF₆⁻, ClO₄⁻SbF₆⁻AsF₆⁻ or an organic anion selected from the group consisting of C₁-C₂₀alkyl-COO⁻, C₆-C₂₀aryl-COO⁻, C₁-C₂₀alkyl-S(═O)₂O⁻, C₆-C₂₀aryl-S(═O)₂O⁻, C₁-C₂₀alkyl-O—S(═O)₂O⁻, C₆-C₂₀aryl-O—S(═O)₂O⁻, C₁-C₂₀alkyl-P(═O)₂O⁻, C₆-C₂₀aryl-P(═O)₂O⁻, C₁-C₂₀alkyl-O—P(═O)₂O⁻ and C₆-C₂₀aryl-O—P(═O)₂O⁻, whereby the aryl is unsubstituted or substituted by 1 to 4 C₁-C₂₀alkyl, or Y⁻ is an anionic group which is part of a compound of formula (I) and is selected from the group consisting of COO⁻, S(═O)₂O⁻, O—S(═O)₂O⁻, P(═O)(O⁻)(O⁻Z⁺), P(═O)(OH)(O⁻), O—P(═O)(O⁻)(O⁻Z⁺), O—P(═O)(OH)(O⁻), —CO(C═O)O⁻ and —NR₁₄—S(═O)₂O⁻;
G and G′ are independently —R₂₈—COOH, —R₂₈—COO⁻Z⁺, —R₂₈—CO(C═O)OH, —R₂₈—CO(C═O)O⁻Z⁺, —R₂₈—S(═O)₂OH, —R₂₈—S(═O)₂O⁻Z⁺, —R₂₈—O—S(═O)₂OH, —R₂₈—O—S(═O)₂O⁻Z⁺, —R₂₈—P(═O)(OH)₂, —R₂₈—P(═O)(O⁻Z⁺)₂, —R₂₈—P(═O)(OH)(O⁻Z⁺), —R₂₈—O—P(═O)(OH)₂, —R₂₈—O—P(═O)(O⁻Z⁺)₂, —R₂₈—O—P(═O)(OH)(O⁻Z⁺), —R₂₈—CO—NH—OH, —R₂₈—S(═O)₂NH—OH, —R₂₈—NR₁₄—S(═O)₂OH or —R₂₈—NR₁₄—S(═O)₂O⁻Z⁺;
Z⁺ is N(R₁₄)₄⁺, Li⁺, Na⁺ or K⁺ or is the cationic group 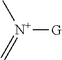
which is part of a compound of formula (I) as part of group A;
R₂₈ is a direct bond or C₁-C₂₀alkylene, C₂-C₂₀alkenylene, C₂-C₂₀alkynylene, C₆-C₂₀arylene, C₄-C₉heteroarylene, C₇-C₁₁aralkylene, C₈-C₁₁ aralkenylene, C₈-C₁₁aralkynylene, C₆-C₁₁heteroaralkylene, C₇-C₁₁heteroaralkenylene, C₇-C₁₁heteroaralkynylene, C₅-C₆cycloalkylene, C₁-C₂₀alkylene-C₆-C₂₀arylene, C₁-C₂₀alkylene-C₄-C₉heteroarylene, C₁-C₂₀alkylene-C₆-C₂₀arylene-C₁-C₂₀alkylene, C₁-C₂₀alkylene-C₄-C₉heteroarylene-C₁-C₂₀alkylene, —O—C₁-C₂₀alkylene, —O—C₂-C₂₀alkenylene, —O—C₂-C₂₀alkynylene, —O—C₆-C₂₀arylene, —O—C₄-C₉heteroarylene, —O—C₇-C₁₁aralkylene, —O—C₈-C₁₁aralkenylene, —O—C₈-C₁₁aralkynylene, —O—C₆-C₁₁heteroaralkylene, —O—C₇-C₁₁heteroaralkenylene, —O—C₇-C₁₁heteroaralkynylene, —O—C₅-C₆cycloalkylene, —O—C₁-C₂₀alkylene-C₆-C₂₀arylene, —O—C₁-C₂₀alkylene-C₄-C₉heteroarylene, —O—C₁-C₂₀alkylene-C₆-C₂₀arylene-C₁-C₂₀alkylene or —O—C₁-C₂₀alkylene-C₄-C₉heteroarylene-C₁-C₂₀alkylene, whereby each of said groups is unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the arylene and heteroarylene can be further substituted by 1-4 C₁-C₂₀alkyl;
R₃₀, R₃₁, R₃₂ or R₃₃ are independently G′, H, halogen, pyridinium*Y⁻, quinolinium*Y⁻, isoquinolinium*Y⁻, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl, whereby the whereby the pyridinium, quinolinium, isoquinolinium, alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by 1-4 C₁-C₂₀alkyl, and whereby the pyridinium, quinolinium and isoquinolinium can be further substituted by G′, C₁-C₂₀alkyl or combinations thereof; or 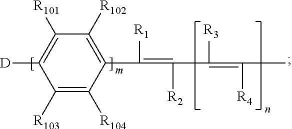
or two vicinal groups of R₃₀-R₃₃ form together trimethylene, tetramethylene or pentamethylene, each of which is unsubstituted or substituted by G′, benzo, R₁₄ or combinations thereof.
with the proviso that at least one of R₃₀-R₃₃ is G′;
R₁₀₁, R₁₀₂, R₁₀₃ and R₁₀₄ are independently H, halogen, C₁-C₂₀alkyl, C₂-C₂₀alkenyl, C₂-C₂₀alkynyl, C₆-C₂₀aryl, C₄-C₂₀heteroaryl, C₇-C₂₀aralkyl, C₈-C₂₀aralkenyl, C₈-C₂₀aralkynyl, C₅-C₂₀cycloalkyl, C₅-C₂₀cycloalkenyl or C₆-C₂₀cycloalkynyl, whereby the alkyl and cycloalkyl are uninterrupted or interrupted by O, S, C(═O), NR₁₄ or combinations thereof, and whereby the alkyl, alkenyl, alkynyl, aryl, heteroaryl, aralkyl, aralkenyl, aralkynyl, cycloalkyl, cycloalkenyl and cycloalkynyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′, S(═O)₂OR₁₄, S(═O)₂O⁻Z⁺ or combinations thereof, and the aryl and heteroaryl can be further substituted by C₁-C₂₀alkyl, C₂-C₂₀alkenyl, C₂-C₂₀alkynyl, C₇-C₂₀aralkyl, C₈-C₂₀aralkenyl, C₈-C₂₀aralkynyl, C₄-C₂₀cycloalkyl, C₅-C₂₀cycloalkenyl, C₆-C₂₀cycloalkynyl or combinations thereof;
or R₁₀₂ or R₁₀₄ is D;
or R₁₀₁ and R₁₅ or R₁₀₁ and R₁₆ form together methylene, ethylene, ethenylene or trimethylene, whereby in each of said groups one or more H-atom can be replaced by C₁-C₂₀alkyl, C₁-C₂₀alkylidene, phenyl or combinations thereof, in each of said groups two geminal H-atom can be replaced by C₁-C₂₀alkylidene, and/or in each of said groups two vicinal H-atoms can be replaced by benzo, trimethylene or tetramethylene;
or the compound of formula (I) is dimeric and R₁₈ is 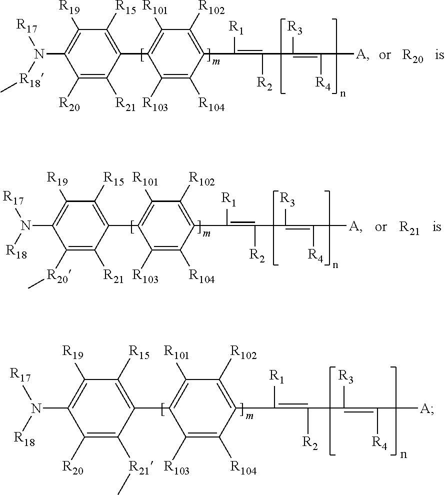
R₁₈′ is R₃₇;
R₂₀′ and R₂₁′ are R₃₇, O—R₃₇—O, S—R₃₇—S, NR₁₄—R₃₇—NR₁₄′, CO—R₃₇—CO, CO—O—R₃₇—O—CO, CO—NR₁₄—R₃₇—NR₁₄′-CO, CO—S—R₃₇—S—CO, O—CO—R₃₇—CO—O, NR₁₄—CO—R₃₇—CO—NR₁₄′ or S—CO—R₃₇—CO—S;
R₃₇ is C₁-C₂₀alkylene, C₂-C₂₀alkenylene, C₂-C₂₀alkynylene, C₆-C₂₀arylene, C₄-C₂₀heteroarylene, C₇-C₂₀aralkylene, C₈-C₂₀aralkenylene, C₈-C₂₀aralkynylene, C₄-C₂₀cycloalkylene, C₁-C₈alkylene-C₆-C₂₀arylene-C₁-C₈alkylene, C₅-C₂₀cycloalkenylene or C₆-C₂₀cycloalkynylene, whereby the alkylene and cycloalkylene is uninterrupted or interrupted by O, S, C(═O), NR₁₄ or combinations thereof, and whereby the alkylene, alkenylene, alkynylene, arylene, heteroarylene, aralkylene, aralkenylene, aralkynylene, cycloalkylene, alkylene-arylene-alkylene, cycloalkenylene and cycloalkynylene are unsubstituted or substituted by fluorine, and whereby the arylene, heteroarylene and aryl can be further substituted by C₁-C₂₀alkyl, fluorinated C₁-C₂₀alkyl or combinations thereof, and the remainder of the substituents are as defined above.

3. An electrode layer according to claim 2, wherein
m is 0 or 1;
n is 0 or 1;
R₁, R₂, R₃ and R₄ are independently H, C₁-C₂₀alkyl or C₆-C₂₀aryl;
or R₁ is D;
R₁₄, R₁₄′ are independently H, C₁-C₁₄alkyl, C₆aryl or C₇-C₁₀aralkyl;
D is a group of formula (III);
R₁₇ and R₁₈ are independently C₁-C₁₄alkyl, C₂-C₈alkenyl, C₆-C₂₀aryl, C₄-C₃₆heteroaryl, C₇-C₂₀aralkyl, C₈-C₂₀aralkenyl, C₈-C₁₀aralkynyl or C₅-C₁₂cycloalkyl, whereby the alkyl, alkenyl, aryl, heteroaryl, aralkyl, aralkenyl, aralkynyl and cycloalkyl, are unsubstituted or substituted by tetrahydrofuranyl, halogen, S—R₁₄, O—R₁₄, CO—OR₁₄, O—CO—R₁₄, NR₁₄R₁₄′, CONR₁₄R₁₄′, NR₁₄—CO—R₁₄′ or combinations thereof, and the aryl and heteroaryl can be further substituted by C₁-C₈alkyl, C₂-C₈alkenyl or C₈-C₂₀aralkenyl;
or R₁₇ and R₁₈ form together with the N they are attached to morpholinyl;
or R₁₇ and R₂₀ and/or R₁₈ and R₁₉ form together with the N-atom R₁₇ and R₁₈ are attached to 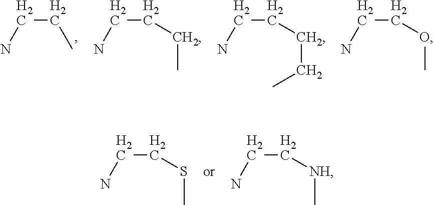
whereby in each of said groups one or more H-atom can be replaced by C₁-C₂₀alkyl, C₆-C₂₀aryl, CO—OR₁₄, CONR₁₄R₁₄′ or combinations thereof, in each of said groups two geminal H-atom can be replaced by C₁-C₂₀alkylidene, and/or in each of said groups two vicinal H-atoms can be replaced by benzo, trimethylene or tetramethylene, whereby the benzo is unsubstituted or substituted by methyl (fluoren-9-ylidene);
R₁₅ is H, NR₂₅R₂₆, OR₂₅, SR₂₅, O—CO—R₂₅ or NR₂₅—CO—R₂₆;
R₁₉, R₂₀ and R₂₁ are H;
R₂₅ and R₂₆ are independently H, C₁-C₁₄alkyl, C₆aryl or C₇-C₁₀aralkyl, whereby the alkyl, aryl and aralkyl are unsubstituted or substituted by halogen, S—R₁₄, O—R₁₄, CO—OR₁₄, O—CO—R₁₄, NR₁₄R₁₄′, CONR₁₄R₁₄′, NR₁₄—CO—R₁₄′ or combinations thereof;
A is a group of formula (V) or (VII),
Y⁻ is Cl⁻, Br⁻, I⁻, SCN⁻, BF₄⁻, PF₆⁻, ClO₄⁻, SbF₆⁻, AsF₆⁻ or an organic anion selected from the group consisting of, C₁-C₂₀alkyl-S(═O)₂O⁻, C₆-C₂₀aryl-S(═O)₂O⁻, C₁-C₂₀alkyl-O—S(═O)₂O⁻, C₆-C₂₀aryl-O—S(═O)₂O⁻, C₁-C₂₀alkyl-P(═O)₂O⁻, C₆-C₂₀aryl-P(═O)₂O⁻, C₁-C₂₀alkyl-O—P(═O)₂O⁻ and C₆-C₂₀aryl-O—P(═O)₂O⁻, whereby the aryl is unsubstituted or substituted by 1 to 4 C₁-C₂₀alkyl, or Y⁻ is an anionic group which is part of a compound of formula (I) and is selected from the group consisting of COO⁻, CO(C═O)O⁻, S(═O)₂O⁻ or OS(═O)₂O⁻;
G and G′ are independently —R₂₈—COOH, —R₂₈—COO⁻Z⁺, —R₂₈—CO(C═O)OH, —R₂₈—CO(C═O)O⁻Z⁺, —R₂₈—S(═O)₂OH, —R₂₈—S(═O)₂O⁻Z⁺, —R₂₈—OS(═O)₂OH, —R₂₈—OS(═O)₂O⁻Z⁺, —R₂₈—CO—NH—OH or —R₂₈—S(═O)₂NH—OH;
Z⁺ is N(R₁₄)₄⁺, Li⁺, Na⁺ or K⁺ or is the cationic group 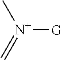
which is part of a compound of formula (I) as part of group A;
R₂₈ is a direct bond or C₁-C₂₀alkylene, C₁-C₂₀alkylene-C₆-C₂₀arylene, —O—C₁-C₂₀alkylene or —O—C₁-C₂₀alkylene-C₆-C₂₀arylene;
R₃₀ is G′;
R₃₁, R₃₂ and R₃₃ are H;
R₁₀₁ and R₁₅ form together methylene, which is substituted by 1 or 2 C₁-C₂₀alkyl;
R₁₀₂, R₁₀₃ and R₁₀₄ are H;
or the compound of formula (I) is dimeric and R₂₁ is 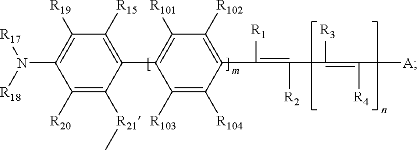
R₂₁′ is O—C₁-C₂₀alkylene-O; and the remainder of the substituents are as defined above.

4. An electrode layer according to claim 3, wherein
m is 0 or 1;
n is 0 or 1;
R₁, R₂, R₃ and R₄ are independently H, C₁-C₁₂alkyl or C₆aryl;
or R₁ is D;
D is a group of formula (III);
R₁₇ and R₁₈ are independently C₁-C₁₄alkyl, C₆aryl, C₄-C₃₀heteroaryl, whereby the aryl is substituted by C₈-C₂₀aralkenyl;
or R₁₇ and R₁₈ form together with the N they are attached to morpholinyl;
or R₁₇ and R₂₀ and/or R₁₈ and R₁₉ form together with the N-atom R₁₇ and R₁₈ are attached to 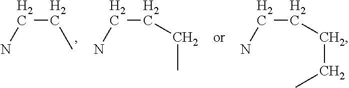
whereby in each of said groups one or more H-atom can be replaced by C₁-C₈alkyl, CON(C₁-C₁₂alkyl)₂, or in each of said groups two vicinal H-atoms can be replaced by benzo or trimethylene;
R₁₅ is H or O—C₁-C₁₄alkyl;
R₁₉, R₂₀ and R₂₁ are H;
A is a group of formula (VII),
Y⁻ is Br⁻, I⁻ or C₁-C₈alkyl-C₆aryl-S(═O)₂O⁻, or Y⁻ is an anionic group which is part of a compound of formula (I) and is selected from the group consisting of COO⁻, OS(═O)₂O⁻ and S(═O)₂O⁻;
G is —R₂₈—COOH, —R₂₈—COO⁻Z⁺, —R₂₈—S(═O)₂OH, —R₂₈—S(═O)₂O⁻Z⁺, —R₂₈—CO—NH—OH or —R₂₈—S(═O)₂NH—OH;
G′ is —COOH, —CO—COOH, —CH₂—COOH or —OS(═O)₂O⁻Z⁺;
Z⁺ is the cationic group 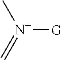
which is part of a compound of formula (I) as part of group A;
R₂₈ is C₁-C₈alkylene, C₁-C₃alkylene-C₆arylene, —O—C₁-C₈alkylene or —O—C₁-C₃alkylene-C₆arylene;
R₃₀ is G′;
R₃₁, R₃₂ and R₃₃ are H;
R₁₀₁ and R₁₅ form together methylene, which is substituted by 2 C₁-C₁₂alkyl;
R₁₀₂, R₁₀₃ and R₁₀₄ are H;
or the compound of formula (I) is dimeric and R₂₁ is 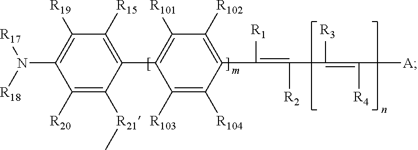
R₂₁′ is O—C₁-C₈alkylene-O; and the remainder of the substituents are as defined above.

5. An electrode layer according to any of claim 1, wherein the oxide semiconductor fine particles are made of TiO₂, SnO₂, WO₃, ZnO, Nb₂O₅, Fe₂O₃, ZrO₂, MgO, WO₃, ZnO, CdS, ZnS, PbS, Bi₂S₃, CdSe, CdTe or combinations thereof.

6. An electrode layer according to any of claim 1, wherein the porous film made of oxide semiconductor fine particles is sensitized with a dye of formula (I) and one or more further dyes.

7. An electrode layer according to claim 6, wherein the further dye is a metal complex dye and/or an organic dye selected from the group consisting of indoline, courmarin, cyanine, merocyanine, hemicyanine, methine, azo, quinone, quinonimine, diketo-pyrrolo-pyrrole, quinacridone, squaraine, triphenylmethane, perylene, indigo, xanthene, eosin, rhodamine and combinations thereof.

8. An electrode layer according to any of claim 1, wherein the dye is adsorbed together with an additive.

9. An electrode layer according to claim 8, wherein the additive is a co-adsorbent selected from a group consisting of a steroid, a sulfonate, a carboxylic acid, a phosphinic acid, a crown ether, a cyclodextrine, a calixarene, a polyethyleneoxide and combinations thereof.

10. A photoelectric conversion device comprising an electrode layer as defined in any of claims 1.

11. A dye sensitized solar cell comprising a photoelectric conversion device as defined in claim 10.

12. A method of using a compound of formula (I) as defined in any of claim 1 as a dye in a dye sensitized solar cell.

13. A compound of formula (I) as defined in claim 1, with the proviso that R₂ and R₁₅ and/or R₂ and R₁₆ do not form together an unsubstituted or substituted aliphatic 5-, 6- or 7-membered ring.
